SEQUENCE LISTING (18 sequences)

SEQ ID NO: 1 - DNA, 21 bp, Artificial Sequence
  Synthetic primer
caagagggaagcagagccttc

SEQ ID NO: 2 - DNA, 24 bp, Artificial Sequence
  Synthetic primer
gcaccttctgaatgtaagagtctc

SEQ ID NO: 3 - DNA, 24 bp, Artificial Sequence
  Synthetic probe
accaaggagcacgcaccacaacga

SEQ ID NO: 4 - DNA, 20 bp, Artificial Sequence
  Synthetic primer
ggccttcgccaccacctatc

SEQ ID NO: 5 - DNA, 22 bp, Artificial Sequence
  Synthetic primer
gtagtacacgtagccctgttcg

SEQ ID NO: 6 - DNA, 24 bp, Artificial Sequence
  Synthetic probe
ccagccatcctcctacccgcagca

SEQ ID NO: 7 - DNA, 22 bp, Artificial Sequence
  Synthetic primer
gctatgtagaggccaagtcctg

SEQ ID NO: 8 - DNA, 20 bp, Artificial Sequence
  Synthetic primer
cagtgacaccccagccattg

SEQ ID NO: 9 - DNA, 24 bp, Artificial Sequence
  Synthetic probe
tcctcctacggtccaggcgaaggc

SEQ ID NO: 10 - DNA, 20 bp, Artificial Sequence
  Synthetic primer
aacggcaagctgtctggaag

SEQ ID NO: 11 - DNA, 22 bp, Artificial Sequence
  Synthetic primer
agccaacattgacctagcactg

SEQ ID NO: 12 - DNA, 24 bp, Artificial Sequence
  Synthetic probe
tcccgcccattcccacccatctcg

SEQ ID NO: 13 - DNA, 24 bp, Artificial Sequence
  Synthetic primer
caagacaggtccagtagagttagg

SEQ ID NO: 14 - DNA, 23 bp, Artificial Sequence
  Synthetic primer
aggtcttataccacctgagcaag

SEQ ID NO: 15 - DNA, 24 bp, Artificial Sequence
  Synthetic probe
acagagcacagccgcctcactgga

SEQ ID NO: 16 - DNA, 21 bp, Artificial Sequence
  Synthetic primer
agaaggtgacctggatgagaa

SEQ ID NO: 17 - DNA, 22 bp, Artificial Sequence
  Synthetic primer
tgatacatatggcggtcaatct

SEQ ID NO: 18 - DNA, 27 bp, Artificial Sequence
  Synthetic probe
cttctcaggagataccgggaatccaag

(19) United States
(12) Patent Application Publication
(10) Pub. No.: US 2010/0203062 A1
(43) Pub. Date: Aug. 12, 2010

(54) Methods and Compositions for Treatment of Neovascularization

(76) Inventors: Ingeborg Stalmans, Leuven (BE); Derek Marshall, Pacifica, CA (US); Tine Van Bergen, Leuven (BE); Victoria Smith, Burlingame, CA (US)

Correspondence Address: Arresto BioSciences;Bozicevic, Field & Francis LLP, 1900 University Ave, Suite 200, East Palo Alto, CA, 94303, US

(21) Appl. No.: 12/701,289

(22) Filed: Feb. 5, 2010

Related U.S. Application Data
(60) Provisional application No. 61/207,202, filed on Feb. 6, 2009.

(51) Int. Cl. A61K 39/395; A61K 31/7052; A61P 9/00; A61P 27/02

(52) U.S. Cl. 424/158.1; 514/44 R

(57) ABSTRACT

Disclosed herein are methods and compositions for treatment of neovascularization, in particular, ocular neovascularization and resultant fibrotic damage. Compositions comprise inhibitors of the activity of one or more lysyl oxidase-type enzymes, and the methods include methods for making the inhibitors and methods for administration of the inhibitors to a subject in need thereof.

### CROSS REFERENCE TO RELATED APPLICATIONS

This application claims the benefit of U.S. Provisional Patent Application No. 61/207,202, filed Feb. 6, 2009; the disclosure of which is incorporated by reference in its entirety for all purposes.

### FIELD

The present application is in the field of ocular neovascularization as occurs, for example, during macular degeneration; and treatments therefor.

### BACKGROUND

Choroidal neovascularization (CNV) refers to abnormal or excessive formation of new blood vessels in the choroid layer of the eye, and is a common symptom of age-related macular degeneration (AMD). In AMD, which is the major cause of irreversible blindness worldwide, CNV is characterized by abnormal growth of choroidal blood vessels through the Bruch's membrane into the subretinal space, leading to inflammation (which generally subsides), angiogenesis, and finally fibrosis in the macula.

Current treatments for AMD and other types of choroidal neovascularization typically involve administration of anti-angiogenic agents. However, such treatments do little to alleviate the inflammation and fibrosis that also result from CNV. Thus, although encouraging in the sense of reversing neovascularization; these treatments are not as clinically effective as might be desired, because they do not address the fibrotic damage resulting from CNV.

Accordingly, anti-fibrotic treatments for ocular neovascularization (e.g., AMD), to be used either separately or in conjunction with anti-angiogenic treatments, would lead to greater clinical success in alleviating vision loss due to CNV.

### SUMMARY

It is disclosed herein that increases in expression of certain lysyl oxidase-type enzymes occur in parallel with the fibrotic damage that follows choroidal neovascularization (CNV). Inhibition of the activity of one or more lysyl oxidase-type enzymes helps to reduce and/or reverse fibrotic damage following CNV. Further, it has been determined that a combination of anti-angiogenic and anti-fibrotic therapies can be used for the treatment of disorders characterized by CNV, for example, age-related macular degeneration (AMD). Anti-fibrotic therapies include inhibition of the activity of one or more lysyl oxidase-type enzymes. Anti-angiogenic therapies include inhibition of the activity of one or more angiogenic factors such as, for example, vascular endothelial growth factor (VEGF).

Compositions for inhibiting the activity of one or more lysyl oxidase-type enzymes and/or inhibiting angiogenesis can comprise proteins, (e.g., antibodies or small peptides), nucleic acids (e.g., triplex-forming oligonucleotides, siRNA, shRNA, microRNA, ribozymes) or small organic molecules (e.g., with a molecular weight of less than 1 kD) as can be synthesized, for example, by combinatorial chemistry.

Thus, the present disclosure includes, but is not limited to, the following embodiments:

1. A method for the treatment of ocular neovascularization in an organism, wherein the method comprises inhibiting the activity of a lysyl oxidase-type enzyme in one or more cells of the organism.

2. The method of embodiment 1, wherein inhibiting comprises binding of an antibody to a lysyl oxidase-type protein.

3. The method of embodiment 2, wherein the lysyl oxidase-type protein is lysyl oxidase (LOX).

4. The method of embodiment 2, wherein the lysyl oxidase-type protein is lysyl oxidase-related protein 2 (LOXL2).

5. The method of embodiment 1, wherein the method further comprises inhibiting the activity of an angiogenic factor in one or more cells of the organism.

6. The method of embodiment 5, wherein the activity of the angiogenic factor is inhibited by binding of an antibody to the angiogenic factor.

7. The method of embodiment 5, wherein the angiogenic factor is a vascular endothelial growth factor (VEGF).

8. The method of embodiment 7, wherein the VEGF is vascular endothelial growth factor A (VEGF-A).

9. The method of embodiment 1, wherein the ocular neovascularization occurs in a disease selected from the group consisting of age-related macular degeneration (AMD), diabetic retinopathy (DR) and retinopathy of prematurity.

10. The method of embodiment 2, wherein the antibody is introduced into the eye of the organism.

11. The method of embodiment 6, wherein the antibodies are introduced into the eye of the organism.

12. The method of embodiment 2, wherein a polynucleotide encoding the antibody is introduced into the eye of the organism.

13. The method of embodiment 6, wherein one or more polynucleotides encoding the antibodies are introduced into the eye of the organism.

14. The method of embodiment 10, wherein the antibody is introduced into one or more retinal epithelial cells.

15. The method of embodiment 11, wherein the antibodies are introduced into one or more retinal epithelial cells.

16. The method of embodiment 12, wherein the polynucleotide is introduced into one or more retinal epithelial cells.

17. The method of embodiment 13, wherein the polynucleotide or polynucleotides are introduced into one or more retinal epithelial cells.

18. The method of embodiment 12, wherein the polynucleotide is encapsidated in a viral vector selected from the group consisting of adeno-associated virus (AAV), adenovirus and lentivirus.

19. The method of embodiment 13, wherein the polynucleotide or polynucleotides are encapsidated in a viral vector selected from the group consisting of adeno-associated virus (AAV), adenovirus and lentivirus.

20. The method of embodiment 18, wherein the viral vector is an adeno-associated virus (AAV).

21. The method of embodiment 19, wherein the viral vector is an adeno-associated virus (AAV).

22. The method of embodiment 20, wherein the viral vector is AAV Type 2 or AAV Type 4.

23. The method of embodiment 21, wherein the viral vector is AAV Type 2 or AAV Type 4.

24. The method of embodiment 1, wherein the organism is a mammal.

25. The method of embodiment 24, wherein the mammal is a human.

### BRIEF DESCRIPTION OF THE DRAWINGS

FIG. 1 shows hematoxylin and eosin (H&E)-stained thin sections of mouse choroid and retina from laser-treated (left and center panels) and control, untreated animals (right panels). The top three photographs show sections of two injured eyes and a control eye at 4 days after laser photocoagulation; the bottom photographs show sections of two injured eyes and one control eye at 7 days after laser photocoagulation. Lesioned sites are enclosed within ovals.

FIG. 2 shows red cyanine 3 immunofluorescence, indicative of CD45 immunoreactivity, in thin sections of mouse choroid and retina, from laser-treated (left and center panels) and control, untreated animals (right panels). The top three photographs show sections of two injured eyes and a control eye at 4 days after laser photocoagulation; the bottom photographs show sections of two injured eyes and one control eye at 7 days after laser photocoagulation. Lesioned sites are enclosed within ovals.

FIG. 3 shows quantitative analysis of levels of CD45-reactive area in sections from control and laser-injured mice at days 14 and 28 after laser photocoagulation. Degree of inflammation is expressed as CD45-positive area as a percent of the total lesion area.

FIG. 4 shows Trichrome-stained thin sections of mouse choroid and retina from laser-treated (left and center panels) and control, untreated animals (right panels). The top three photographs show sections of two injured eyes and a control eye at 4 days after laser photocoagulation; the bottom photographs show sections of two injured eyes and one control eye at 7 days after laser photocoagulation. Lesioned sites are enclosed within ovals.

FIG. 5 shows Sirius Red-stained thin sections of mouse choroid and retina from laser-treated (left and center panels) and control, untreated animals (right panels). The top three photographs show sections of two injured eyes and a control eye at 4 days after laser photocoagulation; the bottom photographs show sections of two injured eyes and one control eye at 7 days after laser photocoagulation. Lesioned sites are enclosed within ovals.

FIG. 6 shows quantitative analysis of collagen deposition in sections from control and laser-injured mice at days 4 and 7 after laser photocoagulation. Collagen deposition was quantitated by determining the area occupied by collagen fibers (staining blue with trichrome and red with Sirius Red) as a percent of the total lesion area. Sirius Red staining was analyzed under polarized light. p=0.00003 for trichrome and 0.00005 for Sirius Red.

FIG. 7 shows quantitative analysis of collagen deposition in sections from control and laser-injured mice at days 14 and 28 after laser photocoagulation. Collagen deposition was quantitated as described in the legend to FIG. 6.

FIG. 8 shows levels of mRNAs encoding lysyl oxidase (LOX) and lysyl oxidase-like (LOXL) proteins in laser injured eyes at 4, 7, 14 and 28 days after photocoagulation. For each of days 4, 7, 14 and 28, each group of five bars represents, from left to right, normalized mRNA levels for LOX, LOXL1, LOXL2, LOXL3 and LOXL4. Bars at each time point represent data for, from left to right, normalized mRNA levels for mLOX, mLOXL1, mLOXL2, mLOXL3 and mLOXL4.

FIG. 9 shows levels of mRNAs encoding lysyl oxidase (LOX) and lysyl oxidase-like (LOXL) proteins in laser injured eyes at 2, 4, 28 and 35 days after photocoagulation. Results were obtained in a separate experiment from the one whose results are depicted in FIG. 8. For each of days 2, 4, 28 and 35, each group of five bars represents, from left to right, normalized mRNA levels for LOX, LOXL1, LOXL2, LOXL3 and LOXL4. Bars at each time point represent data for, from left to right, mLOX, mLOXL1, mLOXL2, mLOXL3 and mLOXL4.

FIG. 10 shows quantitative analysis of levels of CD45-reactive area in sections from laser-injured mouse eyes at day 35 after laser photocoagulation. Mice had been treated with anti-LOXL2 antibody (leftmost bar); anti-LOX antibody (center bar) or vehicle (rightmost bar). Degree of inflammation is expressed as CD45-positive area as a percent of the total lesion area.

FIG. 11 shows quantitative analysis of levels of CD31-reactive area in sections from laser-injured mouse eyes at day 35 after laser photocoagulation. Mice had been treated with anti-LOXL2 antibody (leftmost bar); anti-LOX antibody (center bar) or vehicle (rightmost bar). Degree of neovascularization is expressed as CD31-positive area as a percent of the total lesion area.

FIG. 12 shows quantitative analysis of collagen deposition, by Sirius Red staining, in sections from laser-injured mouse eyes at day 35 after laser photocoagulation. Collagen deposition was quantitated by determining the area occupied by collagen fibers (staining red) as a percent of the total lesion area. Sirius Red staining was analyzed under polarized light.

### DETAILED DESCRIPTION

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N.J., 2000; and the series “Methods in Enzymology,” Academic Press, San Diego, Calif.

Role of Lysyl Oxidase-Type Enzymes in Choroidal Neovascularization

Lysyl oxidase (LOX) and lysyl oxidase-like (LOXL) proteins are involved in the cross-linking of collagen and elastin in the extracellular space. Because of this activity, these proteins can play a major role in the process of fibrosis. It is shown herein that expression of certain lysyl oxidase-type enzymes increases following laser-induced CNV in a model system for age-related macular degeneration (AMD), and that the increases in lysyl oxidase expression parallel the observed fibrotic damage (see Examples 4 and 5 below). Additionally, it shown herein that treatment of subjects with inhibitors of the activity of lysyl oxidase (LOX) and lysyl oxidase-like protein 2 (LOXL2) (e.g., anti-LOX and anti-LOXL2 antibodies) prevents neovascularization and fibrosis following laser-induced CNV in the same system. Accordingly, inhibition of the activity of lysyl oxidase-type enzymes (e.g., LOX, LOXL2) can be used to reverse, mitigate and/or prevent fibrotic damage to the eye resulting from CNV.

Thus, in one aspect, compositions that modulate the activity of one or more lysyl oxidase-type enzymes as described herein are used in the treatment of conditions characterized by neovascularization. A non-limiting example of a condition characterized by neovascularization is age-related macular degeneration (AMD). Additional conditions include diabetic retinopathy and retinopathy of prematurity.

In certain embodiments, an inhibitor of a lysyl oxidase-type enzyme can be an antibody, a small RNA molecule, a ribozyme, a triplex-forming nucleic acid or a transcription factor that inhibits expression of a gene encoding a lysyl oxidase-type protein. See, e.g. US 2006/0127402, US2007/0225242 and co-owned US 2009/0053224; all of which are incorporated by reference for disclosure of various types of lysyl oxidase inhibitors. See also U.S. Pat. No. 6,534,261, incorporated by reference, for disclosure of methods for making transcription factors that inhibit expression of a gene encoding a lysyl oxidase-type enzyme.

In certain embodiments, an inhibitor of a lysyl oxidase-type enzyme is an antibody that binds to, and inhibits the activity of, a lysyl oxidase-type enzyme. In additional embodiments, inhibition is non-competitive. Exemplary antibodies that bind to, and inhibit the activity of, one or more lysyl oxidase-type enzymes are disclosed in co-owned US 2009/0053224; the disclosure of which is incorporated by reference herein for the purpose of disclosing the preparation, composition and use of antibodies that bind to lysyl oxidase-type enzymes.

In certain embodiments, a nucleic acid encoding an antibody, or a functional antibody fragment, is used as an inhibitor of a lysyl oxidase-type enzyme. Such nucleic acids can be administered by any method known in the art. For example, naked nucleic acid, optionally in a buffer or pharmaceutical carrier solution, can be injected into the eye, formulated as a solution for use as eye drops or administered systemically. Alternatively a nucleic acid can be encapsidated in a viral vector (e.g., adenoviral, adeno-associated viral or lentiviral vectors).

Lysyl-Type Enzymes

As used herein, the term “lysyl oxidase-type enzyme” refers to a member of a family of proteins that catalyzes oxidative deamination of ε-amino groups of lysine and hydroxylysine residues, resulting in conversion of peptidyl lysine to peptidyl-α-aminoadipic-δ-semialdehyde (allysine) and the release of stoichiometric quantities of ammonia and hydrogen peroxide:

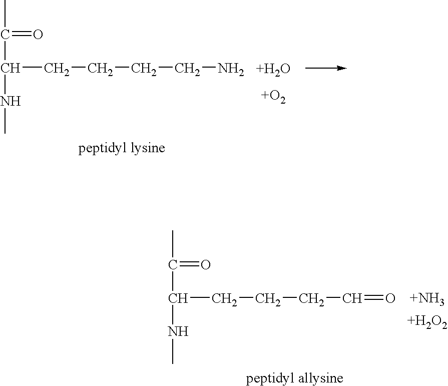

This reaction most often occurs extracellularly, on lysine residues in collagen and elastin. The aldehyde residues of allysine are reactive and can spontaneously condense with other allysine and lysine residues, resulting in crosslinking of collagen molecules to form collagen fibrils.

Lysyl oxidase-type enzymes have been purified from chicken, rat, mouse, bovines and humans. All lysyl oxidase-type enzymes contain a common catalytic domain, approximately 205 amino acids in length, located in the carboxy-terminal portion of the protein and containing the active site of the enzyme. The active site contains a copper-binding site which includes a conserved amino acid sequence containing four histidine residues which coordinate a Cu(II) atom. The active site also contains a lysyltyrosyl quinone (LTQ) cofactor, formed by intramolecular covalent linkage between a lysine and a tyrosine residue (corresponding to lys314 and tyr349 in rat lysyl oxidase, and to lys320 and tyr355 in human lysyl oxidase). The sequence surrounding the tyrosine residue that forms the LTQ cofactor is also conserved among lysyl oxidase-type enzymes. The catalytic domain also contains ten conserved cysteine residues, which participate in the formation of five disulfide bonds. The catalytic domain also includes a fibronectin binding domain. Finally, an amino acid sequence similar to a growth factor and cytokine receptor domain, containing four cysteine residues, is present in the catalytic domain.

The first member of this family of enzymes to be isolated and characterized was lysyl oxidase (EC 1.4.3.13); also known as protein-lysine 6-oxidase, protein-L-lysine:oxygen 6-oxidoreductase (deaminating), or LOX. See, e.g., Harris et al., Biochim. Biophys. Acta 341:332-344 (1974); Rayton et al., J. Biol. Chem. 254:621-626 (1979); Stassen, Biophys. Acta 438:49-60 (1976).

Additional lysyl oxidase-type enzymes were subsequently discovered. These proteins have been dubbed “LOX-like,” or “LOXL.” They all contain the common catalytic domain described above and have similar enzymatic activity. Currently, five different lysyl oxidase-type enzymes are known to exist in both humans and mice: LOX and the four LOX related, or LOX-like proteins LOXL1 (also denoted “lysyl oxidase-like,” “LOXL” or “LOL”), LOXL2 (also denoted “LOR-1”), LOXL3, and LOXL4. The five genes encoding each of the lysyl oxidase-type enzymes each reside on a different chromosome. See, for example, Molnar et al., Biochim Biophys Acta. 1647:220-24 (2003); Csiszar, Prog. Nucl. Acid Res. 70:1-32 (2001); WO 01/83702 published on Nov. 8, 2001, and U.S. Pat. No. 6,300,092, all of which are incorporated by reference herein. A LOX-like protein termed LOXC, with some similarity to LOXL4 but with a different expression pattern, has been isolated from a murine EC cell line. Ito et al. (2001) J. Biol. Chem. 276:24023-24029. Two lysyl oxidase-type enzymes, DmLOXL-1 and DmLOXL-2, have been isolated from Drosophila.

Although all lysyl oxidase-type enzymes share a common catalytic domain, they also differ from one another, particularly within their amino-terminal regions. The four LOXL proteins have amino-terminal extensions, compared to LOX. Thus, while human preproLOX (i.e., the primary translation product prior to signal sequence cleavage, see below) contains 417 amino acid residues; LOXL1 contains 574, LOXL2 contains 638, LOXL3 contains 753 and LOXL4 contains 756.

Within their amino-terminal regions, LOXL2, LOXL3 and LOXL4 contain four repeats of the scavenger receptor cysteine-rich (SRCR) domain. These domains are not present in LOX or LOXL1. SRCR domains are found in secreted, transmembrane, or extracellular matrix proteins, and are known to mediate ligand binding in a number of secreted and receptor proteins. Hoheneste et al. (1999) Nat. Struct. Biol. 6:228-232; Sasaki et al (1998) EMBO J. 17:1606-1613. In addition to its SRCR domains, LOXL3 contains a nuclear localization signal in its amino-terminal region. A proline-rich domain appears to be unique to LOXL1. Molnar et al. (2003) Biochim. Biophys. Acta 1647:220-224. The various lysyl oxidase enzymes also differ in their glycosylation patterns.

Tissue distribution also differs among the lysyl oxidase-type enzymes. Human LOX mRNA is highly expressed in the heart, placenta, testis, lung, kidney and uterus, but marginally in the brain and liver. mRNA for human LOXL1 is expressed in the placenta, kidney, muscle, heart, lung, and pancreas and, similar to LOX, is expressed at much lower levels in the brain and liver. Kim et al. (1995) J. Biol. Chem. 270:7176-7182. High levels of LOXL2 mRNA are expressed in the uterus, placenta, and other organs, but as with LOX and LOXL, low levels are expressed in the brain and liver. Jourdan Le-Saux et al. (1999) J. Biol. Chem. 274:12939:12944. LOXL3 mRNA is highly expressed in the testis, spleen, and prostate, moderately expressed in placenta, and not expressed in the liver, whereas high levels of LOXL4 mRNA are observed in the liver. Huang et al. (2001) Matrix Biol. 20:153-157; Maki and Kivirikko (2001) Biochem. J. 355:381-387; Jourdan Le-Saux et al. (2001) Genomics 74:211-218; Asuncion et al. (2001) Matrix Biol. 20:487-491.

The expression and/or involvement of the different lysyl oxidase-type enzymes in diseases may also vary. See, for example, Kagen (1994) Pathol. Res. Pract. 190:910-919; Murawaki et al. (1991) Hepatology 14:1167-1173; Siegel et al. (1978) Proc. Natl. Acad. Sci. USA 75:2945-2949; Jourdan Le-Saux et al. (1994) Biochem. Biophys. Res. Comm. 199:587-592; and Kim et al. (1999) J. Cell Biochem. 72:181-188. Lysyl oxidase-type enzymes have also been implicated in a number of cancers, including head and neck cancer, bladder cancer, colon cancer, esophageal cancer and breast cancer. See, for example, Wu et al. (2007) Cancer Res. 67:4123-4129; Gorough et al. (2007) J. Pathol. 212:74-82; Csiszar (2001) Prog. Nucl. Acid Res. 70:1-32 and Kirschmann et al. (2002) Cancer Res. 62:4478-4483.

Thus, although the lysyl oxidase-type enzymes exhibit some overlap in structure and function, each appears to have distinct structures and functions as well. For example, targeted deletion of LOX appears to be lethal at parturition in mice, whereas LOXL1 deficiency causes no severe developmental phenotype. Hornstra et al. (2003) J. Biol. Chem. 278:14387-14393; Bronson et al. (2005) Neurosci. Lett. 390:118-122.

Although the most widely documented activity of lysyl oxidase-type enzymes is the oxidation of specific lysine residues in collagen and elastin outside of the cell, there is evidence that lysyl oxidase-type enzymes also participate in a number of intracellular processes. For example, there are reports that some lysyl oxidase-type enzymes regulate gene expression. Li et al. (1997) Proc. Natl. Acad. Sci. USA 94:12817-12822; Giampuzzi et al. (2000) J. Biol. Chem. 275:36341-36349. In addition, LOX has been reported to oxidize lysine residues in histone H1. Additional extracellular activities of LOX include the induction of chemotaxis of monocytes, fibroblasts and smooth muscle cells. Lazarus et al. (1995) Matrix Biol. 14:727-731; Nelson et al. (1988) Proc. Soc. Exp. Biol. Med. 188:346-352. Expression of LOX itself is induced by a number of growth factors and steroids such as TGF-β, TNF-α and interferon. Csiszar (2001) Prog. Nucl. Acid Res. 70:1-32. Recent studies have attributed other roles to LOX in diverse biological functions such as developmental regulation, tumor suppression, cell motility, and cellular senescence.

Examples of lysyl oxidase-type proteins from various sources include enzymes having an amino acid sequence substantially identical to a polypeptide expressed or translated from one of the following sequences: EMBL/GenBank accessions: M94054; AAA59525.1—mRNA; 545875; AAB23549.1—mRNA; 578694; AAB21243.1—mRNA; AF039291; AAD02130.1—mRNA; BC074820; AAH74820.1—mRNA; BC074872; AAH74872.1—mRNA; M84150; AAA59541.1—Genomic DNA. One embodiment of LOX is human lysyl oxidase (hLOX) preproprotein.

Exemplary disclosures of sequences encoding lysyl oxidase-like enzymes are as follows: LOXL1 is encoded by mRNA deposited at GenBank/EMBL BC015090; AAH15090.1; LOXL2 is encoded by mRNA deposited at GenBank/EMBL U89942; LOXL3 is encoded by mRNA deposited at GenBank/EMBL AF282619; AAK51671.1; and LOXL4 is encoded by mRNA deposited at GenBank/EMBL AF338441; AAK71934.1.

The primary translation product of the LOX protein, known as the prepropeptide, contains a signal sequence extending from amino acids 1-21. This signal sequence is released intracellularly by cleavage between Cys21 and Ala22, in both mouse and human LOX, to generate a 46-48 kDa propeptide form of LOX, also referred to herein as the full-length form. The propeptide is N-glycosylated during passage through the Golgi apparatus to yield a 50 kDa protein, then secreted into the extracellular environment. At this stage, the protein is catalytically inactive. A further cleavage, between Gly168 and Asp169 in mouse LOX, and between Gly174 and Asp175 in human LOX, generates the mature, catalytically active, 30-32 kDA enzyme, releasing a 18 kDa propeptide. This final cleavage event is catalyzed by the metalloendoprotease procollagen C-proteinase, also known as bone morphogenetic protein-1 (BMP-1). Interestingly, this enzyme also functions in the processing of LOX's substrate, collagen. The N-glycosyl units are subsequently removed.

Potential signal peptide cleavage sites have been predicted at the amino termini of LOXL1, LOXL2, LOXL3, and LOXL4. The predicted signal cleavage sites are between Gly25 and Gln26 for LOXL, between Ala25 and Gln26, for LOXL2, between Gly25 and Ser26 for LOXL3 and between Arg23 and Pro24 for LOXL4.

A BMP-1 cleavage site in the LOXL (LOXL1) protein has been identified between Ser354 and Asp355. Borel et al. (2001) J. Biol. Chem. 276:48944-48949. Potential BMP-1 cleavage sites in other lysyl oxidase-type enzymes have been predicted, based on the consensus sequence for BMP-1 cleavage in procollagens and pro-LOX being at an Ala/Gly-Asp sequence, often followed by an acidic or charged residue. A predicted BMP-1 cleavage site in LOXL3 is located between Gly447 and Asp448; processing at this site may yield a mature peptide of similar size to mature LOX. A potential cleavage site for BMP-1 was also identified within LOXL4, between residues Ala569 and Asp570. Kim et al. (2003) J. Biol. Chem. 278:52071-52074. LOXL2 may also be proteolytically cleaved analogously to the other members of the LOXL family and secreted. Akiri et al. (2003) Cancer Res. 63:1657-1666.

For the purposes of the present disclosure, the term “lysyl oxidase-type enzyme” encompasses all five of the lysine oxidizing enzymes discussed above, and also encompasses functional fragments and/or derivatives of LOX, LOXL1, LOXL2, LOXL3 and LOXL4 that substantially retain enzymatic activity; e.g., the ability to catalyze deamination of lysyl residues. Typically, a functional fragment or derivative retains at least 50% of its lysine oxidation activity. In some embodiments, a functional fragment or derivative retains at least 60%, at least 70%, at least 80%, at least 90%, at least 95%, at least 99% or 100% of its lysine oxidation activity.

It is also intended that a functional fragment of a lysyl oxidase-type enzyme can include conservative amino acid substitutions (with respect to the native polypeptide sequence) that do not substantially alter catalytic activity. The term “conservative amino acid substitution” refers to grouping of amino acids on the basis of certain common structures and/or properties. With respect to common structures, amino acids can be grouped into those with non-polar side chains (glycine, alanine, valine, leucine, isoleucine, methionine, proline, phenylalanine and tryptophan), those with uncharged polar side chains (serine, threonine, asparagine, glutamine, tyrosine and cysteine) and those with charged polar side chains (lysine, arginine, aspartic acid, glutamic acid and histidine). A group of amino acids containing aromatic side chains includes phenylalanine, tryptophan and tyrosine. Heterocyclic side chains are present in proline, tryptophan and histidine. Within the group of amino acids containing non-polar side chains, those with short hydrocarbon side chains (glycine, alanine, valine, leucine, isoleucine) can be distinguished from those with longer, non-hydrocarbon side chains (methionine, proline, phenylalanine, tryptophan). Within the group of amino acids with charged polar side chains, the acidic amino acids (aspartic acid, glutamic acid) can be distinguished from those with basic side chains (lysine, arginine and histidine).

A functional method for defining common properties of individual amino acids is to analyze the normalized frequencies of amino acid changes between corresponding proteins of homologous organisms (Schulz, G. E. and R. H. Schirmer, Principles of Protein Structure, Springer-Verlag, 1979). According to such analyses, groups of amino acids can be defined in which amino acids within a group are preferentially substituted for one another in homologous proteins, and therefore have similar impact on overall protein structure (Schulz & Schirmer, supra). According to this type of analysis, the following groups of amino acids that can be conservatively substituted for one another can be identified:

(i) amino acids containing a charged group, consisting of Glu, Asp, Lys, Arg and His,

(ii) amino acids containing a positively-charged group, consisting of Lys, Arg and His,

(iii) amino acids containing a negatively-charged group, consisting of Glu and Asp,

(iv) amino acids containing an aromatic group, consisting of Phe, Tyr and Trp,

(v) amino acids containing a nitrogen ring group, consisting of His and Trp,

(vi) amino acids containing a large aliphatic non-polar group, consisting of Val, Leu and Ile,

(vii) amino acids containing a slightly-polar group, consisting of Met and Cys,

(viii) amino acids containing a small-residue group, consisting of Ser, Thr, Asp, Asn, Gly, Ala, Glu, Gln and Pro,

(ix) amino acids containing an aliphatic group consisting of Val, Leu, Be, Met and Cys, and

(x) amino acids containing a hydroxyl group consisting of Ser and Thr.

Thus, as exemplified above, conservative substitutions of amino acids are known to those of skill in this art and can be made generally without altering the biological activity of the resulting molecule. Those of skill in this art also recognize that, in general, single amino acid substitutions in non-essential regions of a polypeptide do not substantially alter biological activity. See, e.g., Watson, et al., “Molecular Biology of the Gene,” 4th Edition, 1987, The Benjamin/Cummings Pub. Co., Menlo Park, Calif., p. 224.

For additional information regarding lysyl oxidase-type enzymes, see, e.g., Rucker et al., Am. J. Clin. Nutr. 67:996 S-1002S (1998) and Kagan et al., J. Cell. Biochem 88:660-672 (2003). See also co-owned US 2009/0053224 (Feb. 26, 2009) and US 2009/0104201 (Apr. 23, 2009); the disclosures of which are incorporated by reference herein.

Modulators of Lysyl Oxidase-Type Enzymes

Modulators of lysyl oxidase-type enzymes include both activators (agonists) and inhibitors (antagonists), and can be selected by using a variety of screening assays. In one embodiment, modulators can be identified by determining if a test compound binds to a lysyl oxidase-type enzyme; wherein, if binding has occurred, the compound is a candidate modulator. Optionally, additional tests can be carried out on such a candidate modulator. Alternatively, a candidate compound can be contacted with a lysyl oxidase-type enzyme, and a biological activity of the lysyl oxidase-type enzyme assayed; a compound that alters the biological activity of the lysyl oxidase-type enzyme is a modulator of a lysyl oxidase-type enzyme. Generally, a compound that reduces a biological activity of a lysyl oxidase-type enzyme is an inhibitor of the enzyme. In certain embodiments, the biological activity is deamination; in additional embodiments, it is peroxide production.

Other methods for identifying modulators of lysyl oxidase-type enzymes include incubating a candidate compound in a cell culture containing one or more lysyl oxidase-type enzymes and assaying one or more biological activities or characteristics of the cells. Compounds that alter the biological activity or characteristic of the cells in the culture are potential modulators of lysyl oxidase-type enzymes. Biological activities that can be assayed include, for example, lysyl oxidase enzymatic activity (e.g., deamination, peroxide production), levels of lysyl oxidase-type enzyme, levels of mRNA encoding one or more lysyl oxidase-type enzymes, and/or one or more functions specific to a lysyl oxidase-type enzyme. In additional embodiments of the aforementioned assay, in the absence of contact with the candidate compound, the one or more biological activities or cell characteristics are correlated with levels or activity of a lysyl oxidase-type enzyme. For example, the biological activity can be a cellular function such as migration, chemotaxis, epithelial-to-mesenchymal transition, or mesenchymal-to-epithelial transition, and the change is detected by comparison with one or more control or reference sample(s). For example, negative control samples can include a culture with decreased levels or activity of a lysyl oxidase-type enzyme to which the candidate compound is added; or a culture with the same amount of lysyl oxidase-type enzyme activity as the test culture, but without addition of candidate compound. In some embodiments, separate cultures containing different levels of a lysyl oxidase-type enzyme are contacted with a candidate compound. If a change in biological activity is observed, and if the change is greater in the culture having higher levels or activity of a lysyl oxidase-type enzyme, the compound is identified as a modulator of a lysyl oxidase-type enzyme. Determination of whether the compound is an activator or an inhibitor of a lysyl oxidase-type enzyme may be apparent from the phenotype induced by the compound, or may require further assay, such as a test of the effect of the compound on lysyl oxidase enzymatic activity.

Methods for obtaining lysysl oxidase-type enzymes, either biochemically or recombinantly, as well as methods for cell culture and enzymatic assay to identify modulators of lysyl oxidase-type enzymes as described above, are known in the art.

The enzymatic activity of a lysyl oxidase-type enzyme can be assayed by a number of different methods. For example, enzymatic activity can be assessed by detecting and/or quantitating production of hydrogen peroxide, ammonium ion, and/or aldehyde, by assaying lysine oxidation and/or collagen crosslinking, or by measuring cellular invasive capacity, cell adhesion, cell growth or metastatic growth. See, for example, Trackman et al. (1981) Anal. Biochem. 113:336-342; Kagan et al. (1982) Meth. Enzymol. 82A:637-649; Palamakumbura et al. (2002) Anal. Biochem. 300:245-251; Albini et al. (1987) Cancer Res. 47:3239-3245; Kamath et al. (2001) Cancer Res. 61:5933-5940; U.S. Pat. No. 4,997,854 and U.S. patent application publication No. 2004/0248871.

Test compounds include, but are not limited to, small organic compounds (e.g., organic molecules having a molecular weight between about 50 and about 2,500 Da), nucleic acids and proteins, for example. The compound or plurality of compounds can be chemically synthesized or microbiologically produced and/or comprised in, for example, samples, e.g., cell extracts from, e.g., plants, animals or microorganisms. Furthermore, the compound(s) can be known in the art but hitherto not known to be capable of modulating a lysyl oxidase-type enzyme. The reaction mixture for assaying for a modulator of a lysyl oxidase-type enzyme can be a cell-free extract or can comprise a cell culture or tissue culture. A plurality of compounds can be, e.g., added to a reaction mixture, added to a culture medium, injected into a cell or administered to a transgenic animal. The cell or tissue employed in the assay can be, for example, a bacterial cell, a fungal cell, an insect cell, a vertebrate cell, a mammalian cell, a primate cell, a human cell or can comprise or be obtained from a non-human transgenic animal.

Several methods are known to the person skilled in the art for producing and screening large libraries to identify compounds having specific affinity for a target, such as a lysyl oxidase-type enzyme. These methods include the phage-display method in which randomized peptides are displayed from phage and screened by affinity chromatography using an immobilized receptor. See, e.g., WO 91/17271, WO 92/01047, and U.S. Pat. No. 5,223,409. In another approach, combinatorial libraries of polymers immobilized on a solid support (e.g., a “chip”) are synthesized using photolithography. See, e.g., U.S. Pat. No. 5,143,854, WO 90/15070 and WO 92/10092. The immobilized polymers are contacted with a labeled receptor (e.g., a lysyl oxidase-type enzyme) and the support is scanned to determine the location of label, to thereby identify polymers binding to the receptor.

The synthesis and screening of peptide libraries on continuous cellulose membrane supports that can be used for identifying binding ligands of a polypeptide of interest (e.g., a lysyl oxidase-type enzyme) is described, for example, in Kramer (1998) Methods Mol. Biol. 87: 25-39. Ligands identified by such an assay are candidate modulators of the protein of interest, and can be selected for further testing. This method can also be used, for example, for determining the binding sites and the recognition motifs in a protein of interest. See, for example Rudiger (1997) EMBO J. 16:1501-1507 and Weiergraber (1996) FEBS Lett. 379:122-126.

WO 98/25146 describes additional methods for screening libraries of complexes for compounds having a desired property, e.g., the capacity to agonize, bind to, or antagonize a polypeptide or its cellular receptor. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with a lysyl oxidase-type enzyme are, for example, in vitro screening with a phage display system, filter binding assays, and “real time” measuring of interaction using, for example, the BIAcore apparatus (Pharmacia).

All these methods can be used in accordance with the present disclosure to identify activators/agonists and inhibitors/antagonists of lysyl oxidase-type enzymes or related polypeptides.

Another approach to the synthesis of modulators of lysyl oxidase-type enzymes is to use mimetic analogs of peptides. Mimetic peptide analogues can be generated by, for example, substituting stereoisomers, i.e. D-amino acids, for naturally-occurring amino acids; see e.g., Tsukida (1997) J. Med. Chem. 40:3534-3541. Furthermore, pro-mimetic components can be incorporated into a peptide to reestablish conformational properties that may be lost upon removal of part of the original polypeptide. See, e.g., Nachman (1995) Regul. Pept. 57:359-370.

Another method for constructing peptide mimetics is to incorporate achiral o-amino acid residues into a peptide, resulting in the substitution of amide bonds by polymethylene units of an aliphatic chain. Banerjee (1996) Biopolymers 39:769-777. Superactive peptidomimetic analogues of small peptide hormones in other systems have been described. Zhang (1996) Biochem. Biophys. Res. Commun. 224:327-331.

Peptide mimetics of a modulator of a lysyl oxidase-type enzyme can also be identified by the synthesis of peptide mimetic combinatorial libraries through successive amide alkylation, followed by testing of the resulting compounds, e.g., for their binding and immunological properties. Methods for the generation and use of peptidomimetic combinatorial libraries have been described. See, for example, Ostresh, (1996) Methods in Enzymology 267:220-234 and Dorner (1996) Bioorg. Med. Chem. 4:709-715. Furthermore, a three-dimensional and/or crystallographic structure of one or more lysyl oxidase enzymes can be used for the design of peptide mimetic inhibitors of lysyl oxidase activity. Rose (1996) Biochemistry 35:12933-12944; Rutenber (1996) Bioorg. Med. Chem. 4:1545-1558.

The structure-based design and synthesis of low-molecular-weight synthetic molecules that mimic the activity of native biological polypeptides is further described in, e.g., Dowd (1998) Nature Biotechnol. 16:190-195; Kieber-Emmons (1997) Current Opinion Biotechnol. 8:435-441; Moore (1997) Proc. West Pharmacol. Soc. 40:115-119; Mathews (1997) Proc. West Pharmacol. Soc. 40:121-125; and Mukhija (1998) European J. Biochem. 254:433-438.

It is also well known to the person skilled in the art that it is possible to design, synthesize and evaluate mimetics of small organic compounds that, for example, can act as a substrate or ligand of a lysyl oxidase-type enzyme. For example, it has been described that D-glucose mimetics of hapalosin exhibited similar efficiency as hapalosin in antagonizing multidrug resistance assistance-associated protein in cytotoxicity. Dinh (1998) J. Med. Chem. 41:981-987.

The structure of the lysyl oxidase-type enzymes can be investigated to guide the selection of modulators such as, for example, small molecules, peptides, peptide mimetics and antibodies. Structural properties of the lysyl oxidase-type enzymes can help to identify natural or synthetic molecules that bind to, or function as a ligand, substrate, binding partner or the receptor of, a lysyl oxidase-type enzyme. See, e.g., Engleman (1997) J. Clin. Invest. 99:2284-2292. For example, folding simulations and computer redesign of structural motifs of lysyl oxidase-type enzymes can be performed using appropriate computer programs. Olszewski (1996) Proteins 25:286-299; Hoffman (1995) Comput. Appl. Biosci. 11:675-679. Computer modeling of protein folding can be used for the conformational and energetic analysis of detailed peptide and protein structure. Monge (1995) J. Mol. Biol. 247:995-1012; Renouf (1995) Adv. Exp. Med. Biol. 376:37-45. Appropriate programs can be used for the identification of sites, on lysyl oxidase-type enzymes, that interact with ligands and binding partners, using computer assisted searches for complementary peptide sequences. Fassina (1994) Immunomethods 5:114-120. Additional systems for the design of protein and peptides are described, for example in Berry (1994) Biochem. Soc. Trans. 22:1033-1036; Wodak (1987), Ann. N.Y. Acad. Sci. 501:1-13; and Pabo (1986) Biochemistry 25:5987-5991. The results obtained from the above-described structural analyses can be used for, e.g., the preparation of organic molecules, peptides and peptide mimetics that function as modulators of the activity of a lysyl oxidase-type enzyme.

An inhibitor of a lysyl oxidase-type enzyme can be a competitive inhibitor, an uncompetitive inhibitor, a mixed inhibitor or a non-competitive inhibitor. Competitive inhibitors often bear a structural similarity to substrate, usually bind to the active site, and are more effective at lower substrate concentrations. The apparent K_{M} is increased in the presence of a competitive inhibitor. Uncompetitive inhibitors generally bind to the enzyme-substrate complex or to a site that becomes available after substrate is bound at the active site and may distort the active site. Both the apparent K_{M} and the Vₘₐₓ are decreased in the presence of an uncompetitive inhibitor, and substrate concentration has little or no effect on inhibition. Mixed inhibitors are capable of binding both to free enzyme and to the enzyme-substrate complex and thus affect both substrate binding and catalytic activity. Non-competitive inhibition is a special case of mixed inhibition in which the inhibitor binds enzyme and enzyme-substrate complex with equal avidity, and inhibition is not affected by substrate concentration. Non-competitive inhibitors generally bind to enzyme at a region outside the active site. For additional details on enzyme inhibition see, for example, Voet et al. (2008) supra.

Antibodies

In certain embodiments, a modulator of a lysyl oxidase-type enzyme is an antibody. In additional embodiments, an antibody is an inhibitor of the activity of a lysyl oxidase-type enzyme.

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab2, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although at a lower affinity than does the entire Fᵥ fragment.

The “F_{ab}” fragment also contains, in addition to heavy and light chain variable regions, the constant domain of the light chain and the first constant domain (CH₁) of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

In some embodiments, an antibody is a humanized antibody or a human antibody. Humanized antibodies include human immununoglobulins (recipient antibody) in which residues from a complementary determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rat, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different lysyl oxidase-type enzymes, or to two different epitopes on a single lysyl oxidase-type enzyme.

An antibody as disclosed herein can also be an immunoconjugate. Such immunoconjugates comprise an antibody (e.g., to a lysyl oxidase-type enzyme) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope on a particular polypeptide is one that binds to that particular polypeptide or epitope without substantially binding to any other polypeptide or polypeptide epitope. In some embodiments, an antibody of the present disclosure specifically binds to its target with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; in the form of monoclonal antibody, scFv, Fab, or other form of antibody measured at a temperature of about 4° C., 25° C., 37° C. or 42° C.

In certain embodiments, an antibody of the present disclosure binds to one or more processing sites (e.g., sites of proteolytic cleavage) in a lysyl oxidase-type enzyme, thereby effectively blocking processing of the proenzyme or preproenzyme to the catalytically active enzyme, thereby reducing the activity of the lysyl oxidase-type enzyme.

In certain embodiments, an antibody according to the present disclosure binds to human LOX and/or human LOXL2, with a greater binding affinity, for example, 10 times, at least 100 times, or even at least 1000 times greater, than its binding affinity to other lysyl oxidase-type enzymes, e.g., LOXL1, LOXL3, and LOXL4.

Optionally, an antibody according to the present disclosure not only binds to a lysyl oxidase-type enzyme but also reduces or inhibits uptake or internalization of the lysyl oxidase-type enzyme, e.g., via integrin beta 1 or other cellular receptors or proteins. Such an antibody could, for example, bind to extracellular matrix proteins, cellular receptors, and/or integrins.

Exemplary antibodies that recognize lysyl oxidase-type enzymes, and additional disclosure relating to antibodies to lysyl oxidase-type enzymes, is provided in co-owned U.S. Patent Application Publication No. 2009/0053224 (Feb. 26, 2009), the disclosure of which is incorporated by reference.

Polynucleotides for Modulating Expression of Lysyl Oxidase-Type Enzymes

Antisense

Modulation (generally inhibition) of a lysyl oxidase-type enzyme can be effected by down-regulating expression of the lysyl oxidase-type enzyme at either the transcriptional or translational level. One such method of modulation involves the use of antisense oligo- or polynucleotides capable of sequence-specific binding with a mRNA transcript encoding a lysyl oxidase-type enzyme.

Binding of an antisense oligonucleotide (or antisense oligonucleotide analogue) to a target mRNA molecule can lead to the enzymatic cleavage of the hybrid by intracellular RNase H. In certain cases, formation of an antisense RNA-mRNA hybrid can interfere with correct splicing. In both cases, the number of intact, functional target mRNAs, suitable for translation, is reduced or eliminated. In other cases, binding of an antisense oligonucleotide or oligonucleotide analogue to a target mRNA can prevent (e.g., by steric hindrance) ribosome binding, thereby preventing translation of the mRNA.

Antisense oligonucleotides can comprise any type of nucleotide subunit, e.g., they can be DNA, RNA, analogues such as peptide nucleic acids (PNA), or mixtures of the preceding. RNA oligonucleotides form a more stable duplex with a target mRNA molecule, but the unhybridized oligonucleotides are less stable intracellularly than other types of oligonucleotides and oligonucleotide analogues. This can be counteracted by expressing RNA oligonucleotides inside a cell using vectors designed for this purpose. This approach may be used, for example, when attempting to target a mRNA that encodes an abundant and long-lived protein.

Additional considerations can be taken into account when designing antisense oligonucleotides, including: (i) sufficient specificity in binding to the target sequence; (ii) solubility in water; (iii) stability against intra- and extracellular nucleases; (iv) ability to penetrate the cell membrane; and (v) when used to treat an organism, low toxicity.

Algorithms for identifying oligonucleotide sequences with the highest predicted binding affinity for their target mRNA, based on a thermodynamic cycle that accounts for the energy of structural alterations in both the target mRNA and the oligonucleotide, are available. For example, Walton et al. (1999) Biotechnol. Bioeng. 65:1-9 used such a method to design antisense oligonucleotides directed to rabbit β-globin (RBG) and mouse tumor necrosis factor-α (TNF a) transcripts. The same research group has also reported that the antisense activity of rationally selected oligonucleotides against three model target mRNAs (human lactate dehydrogenase A and B and rat gp130) in cell culture proved effective in almost all cases. This included tests against three different targets in two cell types with phosphodiester and phosphorothioate oligonucleotide chemistries.

In addition, several approaches for designing and predicting efficiency of specific oligonucleotides using an in vitro system are available. See, e.g., Matveeva et al. (1998) Nature Biotechnology 16:1374-1375.

An antisense oligonucleotide according to the present disclosure includes a polynucleotide or a polynucleotide analogue of at least 10 nucleotides, for example, between 10 and 15, between 15 and 20, at least 17, at least 18, at least 19, at least 20, at least 22, at least 25, at least 30, or even at least 40 nucleotides. Such a polynucleotide or polynucleotide analogue is able to anneal or hybridize (i.e., form a double-stranded structure on the basis of base complementarity) in vivo, under physiological conditions, with a mRNA encoding a lysyl oxidase-type enzyme.

Antisense oligonucleotides according to the present disclosure can be expressed from a nucleic acid construct administered to a cell or tissue. Optionally, expression of the antisense sequences is controlled by an inducible promoter, such that expression of antisense sequences can be switched on and off in a cell or tissue. Alternatively antisense oligonucleotides can be chemically synthesized and administered directly to a cell or tissue, as part of, for example, a pharmaceutical composition.

Antisense technology has led to the generation of highly accurate antisense design algorithms and a wide variety of oligonucleotide delivery systems, thereby enabling those of ordinary skill in the art to design and implement antisense approaches suitable for downregulating expression of known sequences. For additional information relating to antisense technology, see, for example, Lichtenstein et al., “Antisense Technology: A Practical Approach,” Oxford University Press, 1998.

Small RNA and RNAi

Another method for inhibition of lysyl oxidase-type enzymes is RNA interference (RNAi), an approach which utilizes double-stranded small interfering RNA (siRNA) molecules that are homologous to a target mRNA and lead to its degradation. Carthew (2001) Curr. Opin. Cell. Biol. 13:244-248.

RNA interference is typically a two-step process. In the first step, which is termed as the initiation step, input double-stranded RNA (dsRNA) is digested into 21-23 nucleotide (nt) small interfering RNAs (siRNAs), probably by the action of Dicer, a member of the RNase III family of double-strand-specific ribonucleases, which cleaves double-stranded RNA in an ATP-dependent manner. Input RNA can be delivered, e.g., directly or via a transgene or a virus. Successive cleavage events degrade the RNA to 19-21 by duplexes (siRNA), each with 2-nucleotide 3′ overhangs. Hutvagner et al. (2002) Curr. Opin. Genet. Dev. 12:225-232; Bernstein (2001) Nature 409:363-366.

In the second, effector step, siRNA duplexes bind to a nuclease complex to form the RNA-induced silencing complex (RISC). An ATP-dependent unwinding of the siRNA duplex is required for activation of the RISC. The active RISC (containing a single siRNA and an RNase) then targets the homologous transcript by base pairing interactions and typically cleaves the mRNA into fragments of approximately 12 nucleotides, starting from the 3′ terminus of the siRNA. Hutvagner et al., supra; Hammond et al. (2001) Nat. Rev. Gen. 2:110-119; Sharp (2001) Genes. Dev. 15:485-490.

RNAi and associated methods are also described in Tuschl (2001) Chem. Biochem. 2:239-245; Cullen (2002) Nat. Immunol. 3:597-599; and Brantl (2002) Biochem. Biophys. Acta. 1575:15-25.

An exemplary strategy for synthesis of RNAi molecules suitable for use with the present disclosure, as inhibitors of a lysyl oxidase-type enzyme, is to scan the appropriate mRNA sequence downstream of the start codon for AA dinucleotide sequences. Each AA, plus the downstream (i.e., 3′ adjacent) 19 nucleotides, is recorded as a potential siRNA target site. Target sites in coding regions are preferred, since proteins that bind in untranslated regions (UTRs) of a mRNA, and/or translation initiation complexes, may interfere with binding of the siRNA endonuclease complex. Tuschl (2001) supra. It will be appreciated though, that siRNAs directed at untranslated regions can also be effective, as has been demonstrated in the case wherein siRNA directed at the 5′ UTR of the GAPDH gene mediated about 90% decrease in cellular GAPDH mRNA and completely abolished protein level (www.ambion.com/techlib/tn/91/912.html). Once a set of potential target sites is obtained, as described above, the sequences of the potential target sites are compared to an appropriate genomic database (e.g., human, mouse, rat, etc.) using a sequence alignment software, (such as the BLAST software available from NCBI at www.ncbi.nlm.nih.gov/BLAST/). Potential target sites that exhibit significant homology to other coding sequences are rejected.

Qualifying target sequences are selected as templates for siRNA synthesis. Selected sequences can include those with low G/C content as these have been shown to be more effective in mediating gene silencing, compared to those with G/C content higher than 55%. Several target sites can be selected along the length of the target gene for evaluation. For better evaluation of the selected siRNAs, a negative control is used in conjunction. Negative control siRNA can include a sequence with the same nucleotide composition as a test siRNA, but lacking significant homology to the genome. Thus, for example, a scrambled nucleotide sequence of the siRNA may be used, provided it does not display any significant homology to any other gene.

The siRNA molecules of the present disclosure can be transcribed from expression vectors which can facilitate stable expression of the siRNA transcripts once introduced into a host cell. These vectors are engineered to express small hairpin RNAs (shRNAs), which are processed in vivo into siRNA molecules capable of carrying out gene-specific silencing. See, for example, Brummelkamp et al. (2002) Science 296:550-553; Paddison et al (2002) Genes Dev. 16:948-958; Paul et al. (2002) Nature Biotech. 20:505-508; Yu et al. (2002) Proc. Natl. Acad. Sci. USA 99:6047-6052.

Small hairpin RNAs (shRNAs) are single-stranded polynucleotides that form a double-stranded, hairpin loop structure. The double-stranded region is formed from a first sequence that is hybridizable to a target sequence, such as a polynucleotide encoding a lysyl oxidase-type enzyme (e.g., a LOX or LOXL mRNA) and a second sequence that is complementary to the first sequence. The first and second sequences form a double stranded region; while the non-base-paired linker nucleotides that lie between the first and second sequences form a hairpin loop structure. The double-stranded region (stem) of the shRNA can comprise a restriction endonuclease recognition site.

A shRNA molecule can have optional nucleotide overhangs, such as 2-bp overhangs, for example, 3′ UU-overhangs. While there may be variation, stem length typically ranges from approximately 15 to 49, approximately 15 to 35, approximately 19 to 35, approximately 21 to 31 bp, or approximately 21 to 29 bp, and the size of the loop can range from approximately 4 to 30 bp, for example, about 4 to 23 bp.

For expression of shRNAs within cells, plasmid vectors can be employed that contain a promoter (e.g., the RNA Polymerase III H1-RNA promoter or the U6 RNA promoter), a cloning site for insertion of sequences encoding the shRNA, and a transcription termination signal (e.g., a stretch of 4-5 adenine-thymidine base pairs). Polymerase III promoters generally have well-defined transcriptional initiation and termination sites, and their transcripts lack poly(A) tails. The termination signal for these promoters is defined by the polythymidine tract, and the transcript is typically cleaved after the second encoded uridine. Cleavage at this position generates a 3′ UU overhang in the expressed shRNA, which is similar to the 3′ overhangs of synthetic siRNAs. Additional methods for expressing shRNA in mammalian cells are described in the references cited above.

An example of a suitable shRNA expression vector is pSUPER™ (Oligoengine, Inc., Seattle, Wash.), which includes the polymerase-III H1-RNA gene promoter with a well defined transcriptional startsite and a termination signal consisting of five consecutive adenine-thymidine pairs. Brummelkamp et al., supra. The transcription product is cleaved at a site following the second uridine (of the five encoded by the termination sequence), yielding a transcript which resembles the ends of synthetic siRNAs, which also contain nucleotide overhangs. Sequences to be transcribed into shRNA are cloned into such a vector such that they will generate a transcript comprising a first sequence complementary to a portion of a mRNA target (e.g., a mRNA encoding a lysyl oxidase-type enzyme), separated by a short spacer from a second sequence comprising the reverse complement of the first sequence. The resulting transcript folds back on itself to form a stem-loop structure, which mediates RNA interference (RNAi).

Another suitable siRNA expression vector encodes sense and antisense siRNA under the regulation of separate pol III promoters. Miyagishi et al. (2002) Nature Biotech. 20:497-500. The siRNA generated by this vector also includes a five thymidine (T5) termination signal.

siRNAs, shRNAs and/or vectors encoding them can be introduced into cells by a variety of methods, e.g., lipofection. Vector-mediated methods have also been developed. For example, siRNA molecules can be delivered into cell using retroviruses. Delivery of siRNA using retroviruses can provide advantages in certain situations, since retroviral delivery can be efficient, uniform and immediately selects for stable “knock-down” cells. Devroe et al. (2002) BMC Biotechnol. 2:15.

Recent scientific publications have validated the efficacy of such short double stranded RNA molecules in inhibiting target mRNA expression and thus have clearly demonstrated the therapeutic potential of such molecules. For example, RNAi has been utilized for inhibition in cells infected with hepatitis C virus (McCaffrey et al. (2002) Nature 418:38-39), HIV-1 infected cells (Jacque et al. (2002) Nature 418:435-438), cervical cancer cells (Jiang et al. (2002) Oncogene 21:6041-6048) and leukemic cells (Wilda et al. (2002) Oncogene 21:5716-5724).

Methods for Modulating Expression of Lysyl Oxidase-Type Enzymes

Another method of modulating the level or activity of a lysyl oxidase-type enzyme is to modulate the expression of its encoding gene, leading to lower levels of lysyl oxidase activity if gene expression is repressed, and higher levels if gene expression is activated. Modulation of gene expression in a cell can be achieved by a number of methods.

For example, oligonucleotides that bind genomic DNA (e.g., regulatory regions of a lysyl oxidase-type gene) by strand displacement or by triple helix-formation can block transcription, thereby preventing expression of a lysyl oxidase-type enzyme. In this regard, the use of so-called “switch back” chemical linking, in which an oligonucleotide recognizes a polypurine stretch on one strand on one strand of its target and a homopurine sequence on the other strand, has been described. Triple helix formation can also be obtained using oligonucleotides containing artificial bases, thereby extending binding conditions with regard to ionic strength and pH.

Modulation of transcription of a lysyl oxidase-type gene can also be achieved, for example, by introducing into the cell a fusion protein comprising a functional domain and a DNA-binding domain, or a nucleic acid encoding such a fusion protein. A functional domain can be, for example, a transcriptional activation domain or a transcriptional repression domain. Exemplary transcriptional activation domains include VP16, VP64 and the p65 subunit of NF-κB; exemplary transcriptional repression domains include KRAB, KOX and v-erbA.

In certain embodiments, the DNA-binding domain portion of such a fusion protein is a sequence-specific DNA-binding domain that binds in or near a gene encoding a lysyl oxidase-type enzyme or its regulatory region. The DNA-binding domain can either naturally bind to a sequence at or near a gene encoding a lysyl oxidase-type enzyme (or its regulatory region), or can be engineered to so bind. For example, the DNA-binding domain can be obtained from a naturally-occurring protein that regulates expression of a lysyl oxidase-type gene. Alternatively, the DNA-binding domain can be engineered to bind to a sequence of choice in or near a lysyl oxidase-type gene or regulatory region.

In this regard, the zinc finger DNA-binding domain is useful, inasmuch as it is possible to engineer zinc finger proteins to bind to any DNA sequence of choice. A zinc finger binding domain comprises one or more zinc finger structures. Miller et al. (1985) EMBO J. 4:1609-1614; Rhodes (1993) Scientific American, February: 56-65; U.S. Pat. No. 6,453,242. Typically, a single zinc finger is about 30 amino acids in length and contains four zinc-coordinating amino acid residues. Structural studies have demonstrated that the canonical (C₂H₂) zinc finger motif contains two beta sheets (held in a beta turn which generally contains two zinc-coordinating cysteine residues) packed against an alpha helix (generally containing two zinc coordinating histidine residues).

Zinc fingers include both canonical C₂H₂ zinc fingers (i.e., those in which the zinc ion is coordinated by two cysteine and two histidine residues) and non-canonical zinc fingers such as, for example, C₃H zinc fingers (those in which the zinc ion is coordinated by three cysteine residues and one histidine residue) and C₄ zinc fingers (those in which the zinc ion is coordinated by four cysteine residues). Non-canonical zinc fingers can also include those in which an amino acid other than cysteine or histidine is substituted for one of these zinc-coordinating residues. See e.g., WO 02/057293 (Jul. 25, 2002) and US 2003/0108880 (Jun. 12, 2003).

Zinc finger binding domains can be engineered to have a novel binding specificity, compared to a naturally-occurring zinc finger protein; thereby allowing the construction of zinc finger binding domains engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. Engineering methods include, but are not limited to, rational design and various types of empirical selection methods.

Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,030,215; 7,067,617; U.S. Patent Application Publication Nos. 2002/0165356; 2004/0197892; 2007/0154989; 2007/0213269; and International Patent Application Publication Nos. WO 98/53059 and WO 2003/016496.

Exemplary selection methods, including phage display, interaction trap, hybrid selection and two-hybrid systems, are disclosed in U.S. Pat. Nos. 5,789,538; 5,925,523; 6,007,988; 6,013,453; 6,140,466; 6,200,759; 6,242,568; 6,410,248; 6,733,970; 6,790,941; 7,029,847 and 7,297,491; as well as U.S. Patent Application Publication Nos. 2007/0009948 and 2007/0009962; WO 98/37186; WO 01/60970 and GB 2,338,237.

Enhancement of binding specificity for zinc finger binding domains has been described, for example, in U.S. Pat. No. 6,794,136 (Sep. 21, 2004). Additional aspects of zinc finger engineering, with respect to inter-finger linker sequences, are disclosed in U.S. Pat. No. 6,479,626 and U.S. Patent Application Publication No. 2003/0119023. See also Moore et al. (2001a) Proc. Natl. Acad. Sci. USA 98:1432-1436; Moore et al. (2001b) Proc. Natl. Acad. Sci. USA 98:1437-1441 and WO 01/53480.

Further details on the use of fusion proteins comprising engineered zinc finger DAN-binding domains are found, for example, in U.S. Pat. Nos. 6,534,261; 6,607,882; 6,824,978; 6,933,113; 6,979,539; 7,013,219; 7,070,934; 7,163,824 and 7,220,719.

Additional methods for modulating the expression of a lysyl oxidase-type enzyme include targeted mutagenesis, either of the gene or of a regulatory region that controls expression of the gene. Exemplary methods for targeted mutagenesis using fusion proteins comprising a nuclease domain and an engineered DNA-binding domain are provided, for example, in U.S. patent application publications 2005/0064474; 2007/0134796; and 2007/0218528.

Formulations, Kits and Routes of Administration

Therapeutic compositions comprising compounds identified as modulators of the level or activity of a lysyl oxidase-type enzyme (e.g., inhibitors of a lysyl oxidase-type enzyme) are also provided. Such compositions typically comprise the modulator and a pharmaceutically acceptable carrier. Supplementary active compounds can also be incorporated into the compositions. Modulators, particularly inhibitors, of lysyl oxidase-type enzyme(s) are useful, for example, in combination with an anti-angiogenic agent, to reduce or eliminate fibrotic damage resulting from neovascularization. Accordingly, therapeutic compositions as disclosed herein can contain both a modulator of the level and/or activity of a lysyl oxidase-type enzyme and an anti-angiogenic agent. In additional embodiments, therapeutic compositions comprise a therapeutically effective amount of a modulator of the level and/or activity of a lysyl oxidase-type enzyme, but do not contain an anti-angiogenic agent, and the compositions are administered separately from the anti-angiogenic agent.

As used herein, the term “therapeutically effective amount” or “effective amount” refers to an amount of a therapeutic agent that when administered alone or in combination with another therapeutic agent to a cell, tissue, or subject (e.g., a mammal such as a human or a non-human animal such as a primate, rodent, cow, horse, pig, sheep, etc.) is effective to prevent or ameliorate the disease condition or the progression of the disease. A therapeutically effective dose further refers to that amount of the compound sufficient to result in full or partial amelioration of symptoms, e.g., treatment, healing, prevention or amelioration of the relevant medical condition, or an increase in rate of treatment, healing, prevention or amelioration of such conditions. A therapeutically effective amount of, for example, an inhibitor of the level and/or activity of a lysyl oxidase-type enzyme varies with the type of disease or disorder, extensiveness of the disease or disorder, and size of the mammal suffering from the disease or disorder.

The therapeutic compositions disclosed herein are useful for, inter alia, reducing fibrotic damage resulting from neovascularization. Accordingly, a “therapeutically effective amount” of a modulator (e.g., inhibitor) of the level and/or activity of a lysyl oxidase-type enzyme is an amount that results in reduction of fibrotic damage resulting from neovascularization, such as occurs during macular degeneration. For example, when the inhibitor of a lysyl oxidase-type enzyme is an antibody and the antibody is administered in vivo, normal dosage amounts may vary from about 10 ng/kg to up to 100 mg/kg of mammal body weight or more per day, for example, about 1 μg/kg/day to 50 mg/kg/day, optionally about 100 μg/kg/day to 20 mg/kg/day, 500 μg/kg/day to 10 mg/kg/day, or 1 mg/kg/day to 10 mg/kg/day, depending upon, e.g., body weight, route of administration, severity of disease, etc.

When a modulator of the level and/or activity of a lysyl oxidase-type enzyme is used in combination with an anti-angiogenic agent, one can also refer to the therapeutically effective dose of the combination, which is the combined amounts of the modulator and the anti-angiogenic agent that result in reduction of fibrotic damage resulting from neovascularization, whether administered in combination, serially or simultaneously. More than one combination of concentrations can be therapeutically effective.

Various pharmaceutical compositions and techniques for their preparation and use are known to those of skill in the art in light of the present disclosure. For a detailed listing of suitable pharmacological compositions and techniques for their administration one may refer to the detailed teachings herein, which may be further supplemented by texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The disclosed therapeutic compositions further include pharmaceutically acceptable materials, compositions or vehicles, such as a liquid or solid filler, diluent, excipient, solvent or encapsulating material, i.e., carriers. These carriers are involved in transporting the subject chemical from one organ, or region of the body, to another organ, or region of the body. Each carrier should be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Another aspect of the present disclosure relates to kits for carrying out the administration of a modulator of the level and/or activity of a lysyl oxidase-type enzyme. Another aspect of the present disclosure relates to kits for carrying out the combined administration of a modulator of the level and/or activity of a lysyl oxidase-type enzyme and an anti-angiogenic agent. In one embodiment, a kit comprises an inhibitor of the activity of a lysyl oxidase-type enzyme formulated in a pharmaceutical carrier, optionally containing at least one anti-angiogenic agent that is not an inhibitor of the activity of a lysyl oxidase-type enzyme, formulated as appropriate, in one or more separate pharmaceutical preparations.

The formulation and delivery methods can be adapted according to the site(s) and degree of fibrotic damage. Exemplary formulations include, but are not limited to, those suitable for parenteral administration, e.g., intravenous, intra-arterial, intra-ocular, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compounds of the disclosure will vary according to the extent and severity of the need for treatment, the activity of the administered composition, the general health of the subject, and other considerations well known to the skilled artisan.

In additional embodiments, the compositions described herein are delivered locally. Such local delivery can be achieved, for example, by intra-ocular injection or by application of eye drops.

Administration

For treatment of choroidal neovascularization (e.g., AMD) with inhibitors of the activity of a lysyl oxidase-type enzyme (e.g., inhibitors of LOX and/or LOXL2 activity), any method known in the art for delivery of substances to the eye can be utilized. For example, direct injection into the eye can be used for delivery of an inhibitor of the activity of a lysyl oxidase-type enzyme; e.g., an anti-LOX antibody and/or an anti-LOXL2 antibody. In certain embodiments, an inhibitor of the level and/or activity of a lysyl oxidase-type enzyme (optionally in combination with an angiogenesis inhibitor, see below) is injected into the vitreous humor. In additional embodiments, topical administration of an inhibitor of the level and/or activity of a lysyl oxidase-type enzyme is used. For example, the eye can be bathed in a solution containing an inhibitor of the level and/or activity of a lysyl oxidase-type enzyme, or an inhibitor of the level and/or activity of a lysyl oxidase-type enzyme can be formulated in a solution to be used as eye drops. An inhibitor of the level and/or activity of a lysyl oxidase-type enzyme can also be administered systemically, provided an effective concentration reaches the eye and there are no (or acceptable) extra-ocular side effects.

Nucleic acids encoding anti-lysyl oxidase antibodies (or any other type of inhibitor of a lysyl oxidase-type enzyme, e.g., a ribozyme, siRNA, shRNA or microRNA) can optionally be encapsidated in a viral vector. A number of viral vectors are known in the art, including parvoviruses, papovaviruses, adenoviruses, herpesviruses, poxviruses, retroviruses and lentiviruses.

One class of recombinant viral vectors is based on the defective and nonpathogenic parvovirus adeno-associated virus serotype 2 (AAV-2). Vectors are derived from a plasmid containing the AAV 145 by inverted terminal repeat sequence flanking a transgene expression cassette. Efficient gene transfer and stable transgene delivery due to integration into the genomes of the infected cell are obtained using this vector system. Wagner et al. (1998) Lancet 351:1702-1703; Kearns et al. (1996) Gene Ther. 9:748-755

Additional adeno-associated virus vehicles include AAV serotypes 1, 5, 6, 7, 8 and 9; as well as chimeric AAV serotypes, e.g., AAV 2/1 and AAV 2/5. Both single-stranded and double-stranded (e.g., self-complementary) AAV vectors can be used.

Combination Therapies

In certain embodiments, treatment of conditions characterized by neovascularization involves administration of a composition as described herein that inhibits the level and/or activity of a lysyl oxidase-type enzyme, together with administration of a second composition that inhibits angiogenesis. The compositions can be administered sequentially in any order or concurrently. In certain embodiments, both compositions comprise antibodies. In other embodiments, both compositions comprise polynucleotides encoding antibodies. In still further embodiments, one composition comprises a polynucleotide encoding an antibody and the other comprises an antibody polypeptide. In embodiments in which the compositions are administered as polynucleotides, a single polynucleotide (e.g., expression vector) can be used that encodes both inhibitors.

In certain embodiments, an inhibitor of angiogenesis is an anti-VEGF antibody. Inhibitors of this type are available commercially, for example, under the trade names Avastin® and Lucentis®. However, any anti-VEGF antibody can be used.

In additional embodiments, an inhibitor of angiogenesis can be a small RNA molecule, a ribozyme, a triplex-forming nucleic acid or a transcription factor that inhibits expression of a VEGF gene. See, e.g., U.S. Pat. No. 7,067,317.

### EXAMPLES

### Example 1

### Mouse Model of AMD

Laser-induced photocoagulation of the retina in mice, leading to CNV, was used as a model system for AMD. This treatment induces ruptures in the Bruch's membrane, induces neovascularization, and forms scar tissue that seals leaky blood vessels. In this model, inflammation was previously observed for up to five days after coagulation, and was followed by angiogenesis with a peak at day 14. Rakic et al. (2003) Invest. Opthalmol. Vis. Sci. 44:3186-3193. At even later stages after photocoagulation (3-4 weeks), fibrosis was observed. In the present example, the degree and extent of inflammation and fibrosis, along with the expression of lysyl oxidase (LOX) and lysyl oxidase-like proteins (LOXLs), in the eye (choroid and retina) after laser photocoagulation, was assessed.

For these experiments, male C57BL/6 mice, at 8-10 weeks of age, were acclimated at 20±2° C., relative humidity of 55±5% and a 14 hour light/10 hour dark cycle, for at least five days, then certain of the animals were subjected to laser photocoagulation, while other animals that did not receive the photocoagulation treatment served as controls.

For photocoagulation, mice were anesthetized with an intrapeitoneal injection of Nembutal™ (60 mg/ml) and the pupils were dilated with topical administration of Tropicol™ (5 mg/ml). Three burns were placed with an Argon laser (532 nm) at 9, 12, and 3 o′ clock positions in each retina using a slit lamp delivery system. The laser was set for a 0.05 second duration at an energy of 400 mW, and a 50 μm spot size. Rupture of the Bruch's membrane was confirmed by production of an air bubble at the site at which the laser had been aimed, and only sites at which a bubble was observed were included in the analysis.

Five animals that had undergone laser injury were sacrificed on days 4 and 7, along with three uninjured control animals. For analyses conducted on days 14 and 28 after photocoagulation, three injured animals and three control animals were sacrificed at each time point.

All animals were sacrificed by cervical dislocation. Immediately after sacrifice, both eyes were enucleated and choroidea and retinas were dissected. One animal from the control group and one from the photocoagulation group, at each of the four time points, was used for analysis of the extent of laser-induced injury, inflammation and fibrosis. Tissue for these analyses was frozen in 4% paraformaldehyde and embedded in paraffin. Seven μm sections were cut. Sections were stained with hematoxylin and eosin to detect the lesions; other sections were tested for CD45 levels (by immunohistochemistry) to evaluate the degree of inflammation, and separate sections were stained with Sirius Red and Trichrome stain to evaluate the degree and extent of fibrosis. Images were obtained with a Zeiss Imager Z1 at a magnification of 10× and a resolution of 1292×968 pixels, and photographs were taken with a Zeiss Axiocam MrC5. Images were morphologically analyzed with Zeiss KS300 software. This software was used to determine the total area of the lesion, to measure areas within the lesion that stained positively for different markers (see below), and to calculate the fraction of the total lesion area positive for the particular marker under study. Data were analyzed with Statistica 6.1 statistical software, using a student T-test for independent samples. P-values smaller than 0.05 were considered statistically significant.

The remaining animals (four from the photocoagulation group on days 4 and 7, two from the photocoagulation group on days 14 and 28, and two controls at each of the four time points) were used for analysis of lysyl oxidase mRNA. For mRNA analysis, fresh tissue (choroid and retina) was frozen in liquid nitrogen and stored at −80° C. until used for RNA extraction (below).

### Example 2

### Lesion Detection

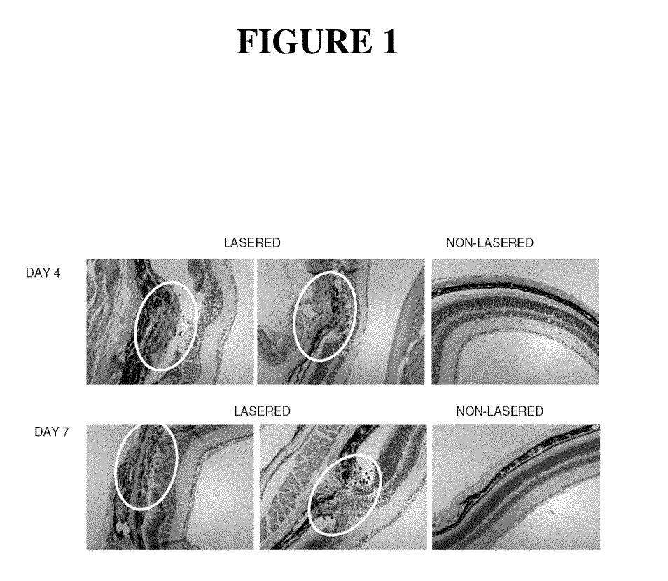

All eyes were examined histologically, using hematoxylin and eosin (H&E)-stained sections. Three lesions were detected on each eye that had been subjected to laser treatment. An example is shown in FIG. 1.

### Example 3

### Inflammation

Thin sections were also subjected to immunohistochemistry for CD45, a leukocyte marker, whose presence is indicative of inflammation. For this analysis, antigen retrieval was conducted for 20 minutes at 95° C., and rabbit serum was used as a blocking agent. Sections were incubated overnight at room temperature with rat anti-mouse CD45 antibody ( 1/100; Beckton Dickinson). The following day the slides were incubated with a biotinylated rabbit anti-rat antibody (Dakocytomation) at a 1/300 dilution for 45 minutes at room temperature. Sections were then developed using a TSA Cyan 3 System (Perkin Elmer TSATM; NEL704A) at room temperature, and washed with TNT washing buffer. Streptavidin peroxidase was used at a 1/100 dilution and cyan 3 was diluted 1/50 in working buffer.

The degree of inflammation was quantitated by determining the area of the section exhibiting CD45 immunoreactivity and expressing this as a percentage of the total area of the lesion. Areas were determined using the Zeiss KS300 software.

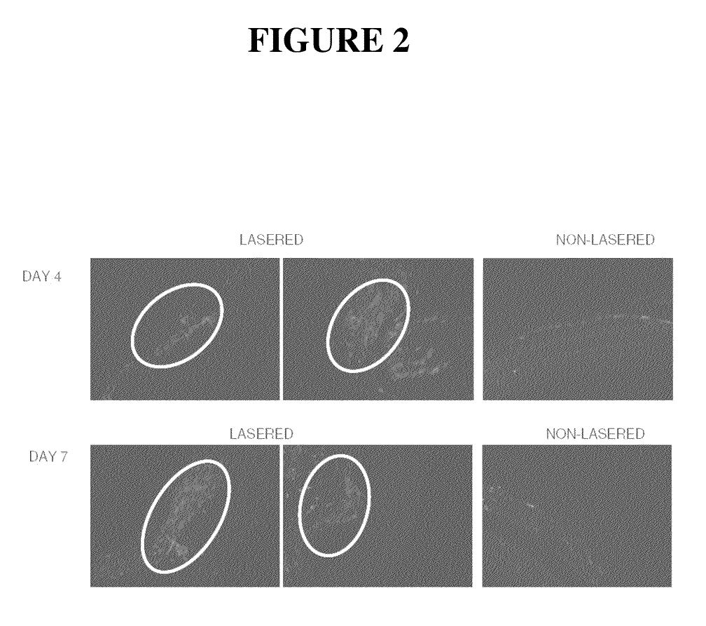

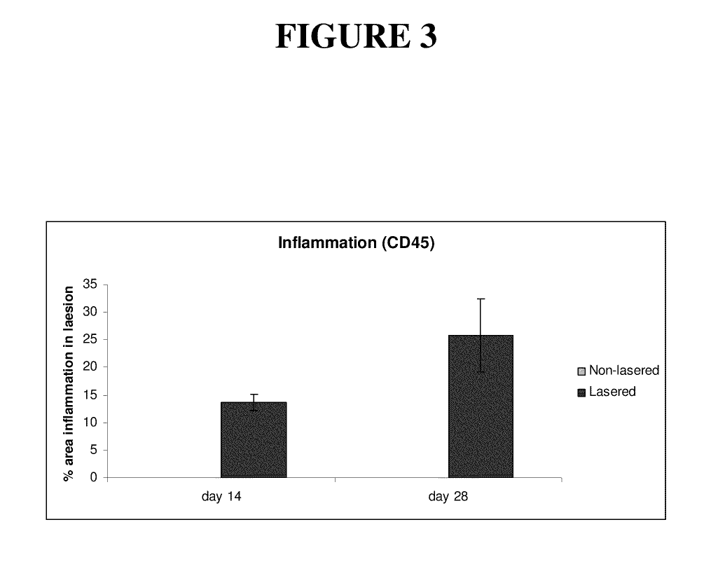

The results of this analysis indicated that no leukocytes were present (i.e., no CD45 immunoreactivity was observed) in untreated eyes. However, inflammation, as evidenced by CD45 immunoreactivity, was apparent in laser-treated eyes as early as day 4 after laser treatment. In laser-injured eyes, CD45 levels remained roughly constant on days 4, 7 and 14 but, by day 28, they had approximately doubled. See FIG. 2 for examples of stained samples. FIG. 3 shows quantitation of CD45 levels on days 14 and 28 after laser injury.

### Example 4

### Fibrosis

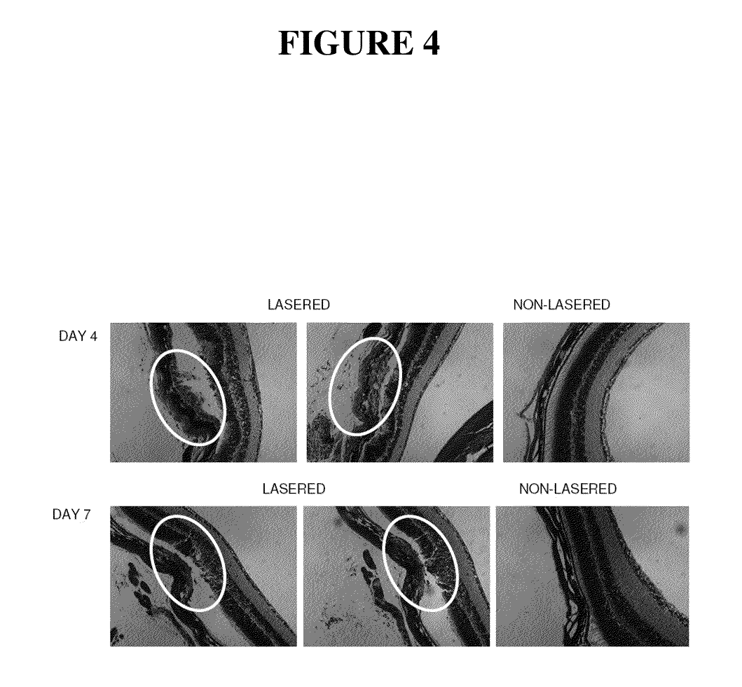

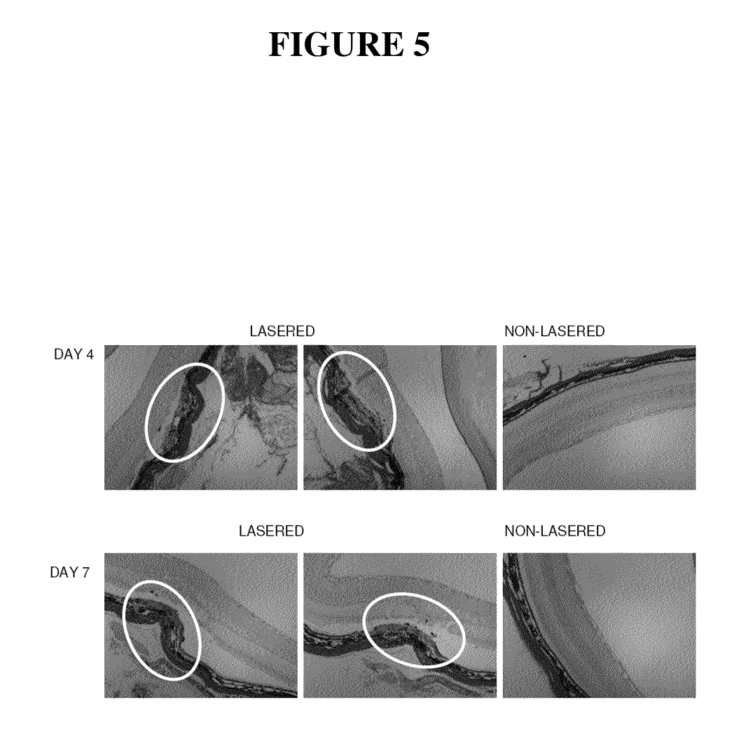

Thin sections, obtained as described above, were stained with Trichrome and Sirius Red, then analyzed by microscopy. Examples of Trichrome-stained and Sirius Red-stained sections, at days 4 and 7 after laser injury, are shown in FIGS. 4 and 5.

The extent of fibrosis was scored quantitatively by determining the area of the section exhibiting collagen staining and expressing this as a percentage of the total area of the lesion. Areas were determined using the Zeiss KS300 software.

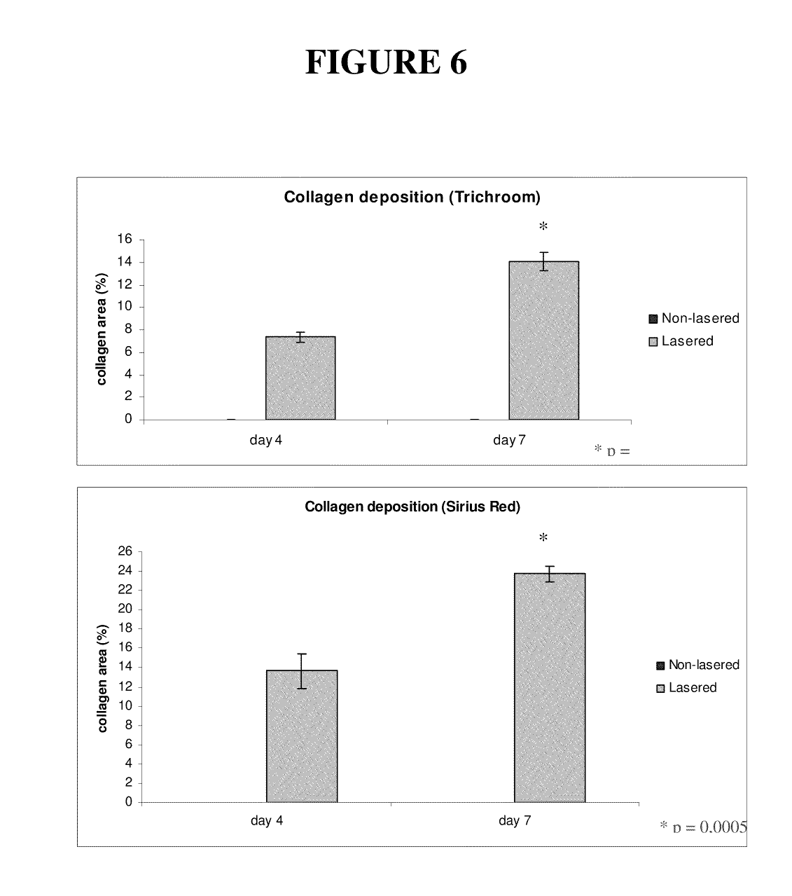

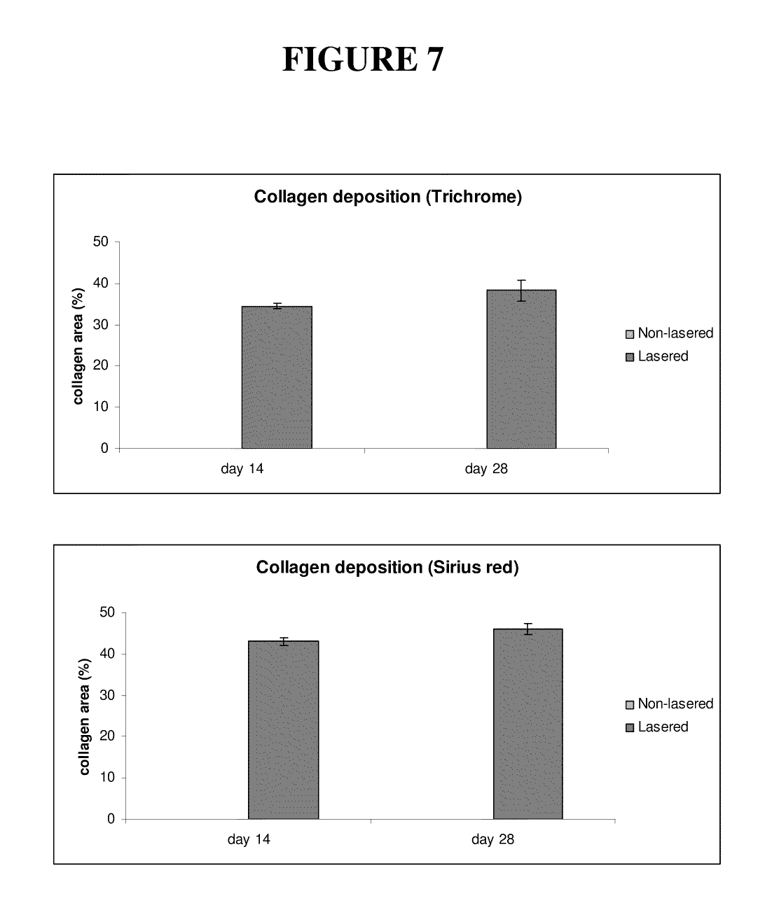

Results of the quantitation did not reveal collagen deposition (indicative of fibrosis) in non-laser-treated eyes. In laser-injured eyes, collagen deposition was observed (by both Trichrome and Sirius Red staining) as early as day 4 after injury, and collagen levels continued to increase through days 7 and 14. Collagen levels at day 28 were approximately equivalent to those observed at day 14. FIG. 6 shows collagen deposition at days 4 and 7, and FIG. 7 shows collagen deposition at days 14 and 28. (Data obtained from two separate experiments.) These results indicate that fibrosis occurs rapidly after laser injury, increases to an apparent plateau at day 14, and lasts at least an additional two weeks thereafter.

### Example 5

### Lysyl Oxidase Expression

RNA was purified from frozen tissue (see Example 1) for analysis of lysyl oxidase (LOX) and lysyl oxidase-related protein (LOXL) transcript levels.

Retina and choroid from each mouse eye were pooled, suspended in 700 μl Qiagen RLT buffer containing β-mercaptoethanol, and homogenized with a Polytron hand-held electric homogenizer. RNA isolation was performed using a RNeasy Mini kit, according to the manufacturer's instructions (Qiagen, Valencia, Calif.). Eluted RNA was DNase-treated with Ambion rDNAse I according to reagent specifications.

Levels of mRNA for lysyl oxidase and lysyl oxidase-like proteins were determined by quantitative reverse transcription/polymerase chain reaction (qRT-PCR). Reverse transcription and amplification reactions were performed using a Stratagene Brilliant II One-Step Core Reagents kit according to the manufacturer's instructions, using 100 ng RNA template in each reaction. Primers and FAM/BHQ-1 probes for target mRNA were designed using Beacon Designer™ software (Premier Biosoft, Palo Alto, Calif.) and were used at final concentrations of 400 nM for primers and 250 nM for probes. Nucleotide sequences of the probes and primers are presented in Table 1.

TABLE 1

Probe and primer sequences for analysis of LOX and LOXL mRNA levels

| | | | |
|---|---|---|---|
| Gene | Forward primer | Reverse primer | Probe |

| | | | |
|---|---|---|---|
| LOX | CAAGAGGGAAGCAGAGCCTTC | GCACCTTCTGAATGTAAGAGTCTC | ACCAAGGAGCACGCACCACAACGA |
| | (SEQ ID NO: 1) | (SEQ ID NO: 2) | (SEQ ID NO: 3) |
| LOXL1 | GGCCTTCGCCACCACCTATC | GTAGTACACGTAGCCCTGTTCG | CCAGCCATCCTCCTACCCGCAGCA |
| | (SEQ ID NO: 4) | (SEQ ID NO: 5) | (SEQ ID NO: 6) |
| LOXL2 | GCTATGTAGAGGCCAAGTCCTG | CAGTGACACCCCAGCCATTG | TCCTCCTACGGTCCAGGCGAAGGC |
| | (SEQ ID NO: 7) | (SEQ ID NO: 8) | (SEQ ID NO: 9) |
| LOXL3 | AACGGCAAGCTGTCTGGAAG | AGCCAACATTGACCTAGCACTG | TCCCGCCCATTCCCACCCATCTCG |
| | (SEQ ID NO: 10) | (SEQ ID NO: 11) | (SEQ ID NO: 12) |
| LOXL4 | CAAGACAGGTCCAGTAGAGTTAGG | AGGTCTTATACCACCTGAGCAAG | ACAGAGCACAGCCGCCTCACTGGA |
| | (SEQ ID NO: 13) | (SEQ ID NO: 14) | (SEQ ID NO: 15) |
| RPL19 | AGAAGGTGACCTGGATGAGAA | TGATACATATGGCGGTCAATCT | CTTCTCAGGAGATACCGGGAATCCAAG |
| | (SEQ ID NO: 16) | (SEQ ID NO: 17) | (SEQ ID NO: 18) |

Primer/probe sets were validated for specificity for their target mRNAs by in vitro siRNA knock-down experiments and were tested for their amplification efficiencies using dilutions of cell line RNA expressing moderate to high levels of target mRNAs. An efficiency of 100% corresponds to a doubling in the amount of amplicon during each cycle that occurs during the exponential phase of the amplification reaction, and results in a 10-fold increase in the amount of amplicon every 3.32 cycles. Efficiency was determined by plotting Cₜ vs. input RNA concentration on a semi-logarithmic scale and determining the slope of the curve so generated. Percent efficiency (E) was then calculated as follows:

E=(10^{-1/slope}−1)×100

The amplification efficiencies of all primer/probe sets were determined to be >90%.

Test mRNA levels were normalized to mRNA levels of ribosomal protein L19 (RPL19), and results were expressed as fold regulation of relative expression in laser-coagulated retina compared to relative expression in control, nontreated retina. Results were based on averages of two experimental (4 retina+4 choroid) and one control (2 retina+2 choroid) animal for each time point.

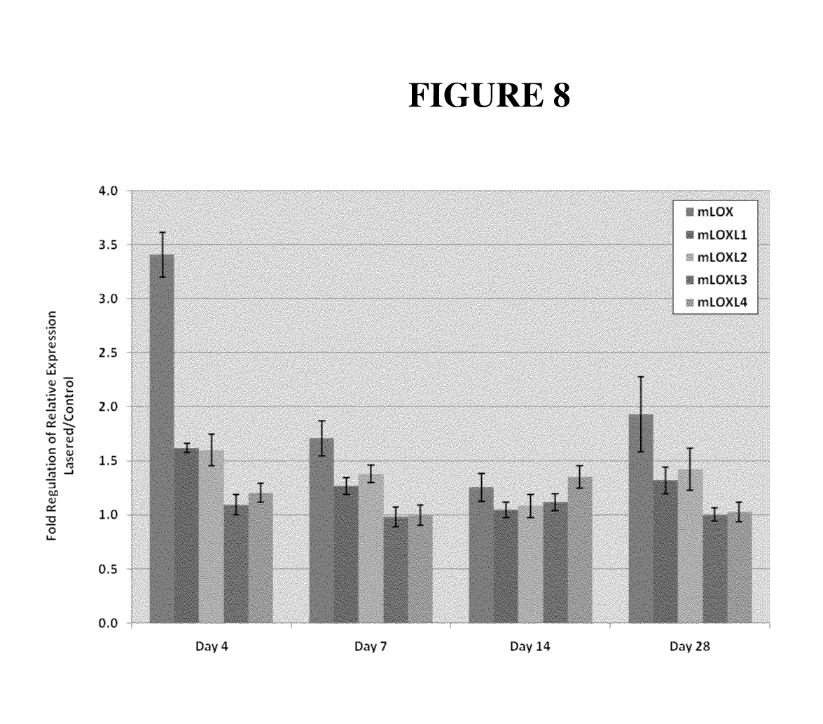

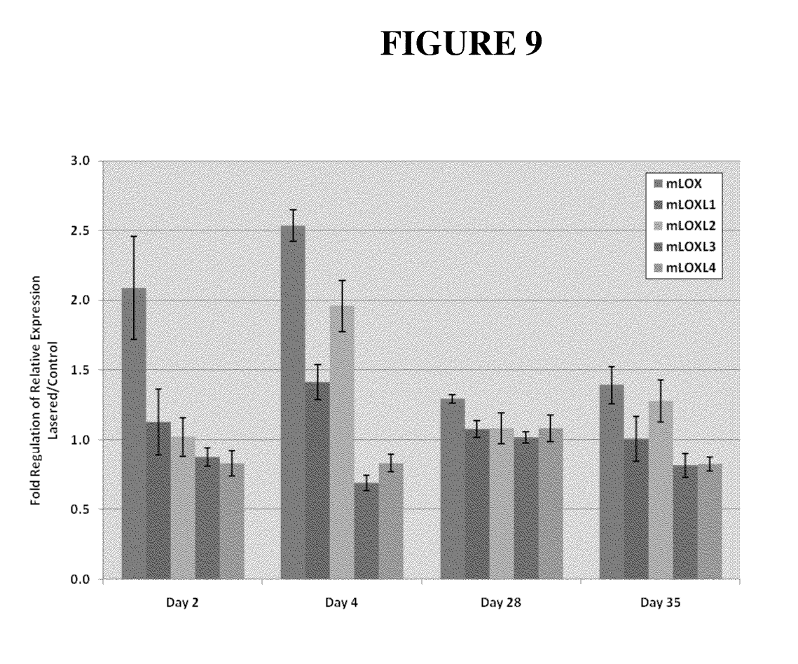

The results of the mRNA analysis (FIG. 8) showed that expression of the lysyl oxidase gene (LOX) is increased over three-fold at day 4 after laser injury. Increases in the levels of mRNAs encoding LOXL1 and LOXL2 were also observed at day 4. In a separate experiment in which RNA from four experimental animals and two control animals was analyzed at each of days 2, 4, 28 and 35 after laser photocoagulation, increases in levels of mRNA for LOX, LOXL1 and LOXL2, at day 4 after injury, were also observed (FIG. 9).

Additional experiments have shown that levels of LOX and LOXL2 remain elevated in photocoagulated eyes for at least 35 days after photocoagulation.

### Example 6

### Inhibition of LOX and LOXL2 Activities Reduces Fiborsis, Inflammation and Neovascularization Associated with Macular Degeneration

In this example, the effect of treatment with antibodies to LOX and LOXL2, in a murine model of age-related macular degeneration, was assessed. In particular, effects on fibrosis, inflammation and angiogenesis were investigated. Anti-LOX antibody M64 and anti-LOXL2 antibody AB0023 are both described in co-owned US Patent Application Publication No. US 2009/0053224 (Feb. 26, 2009) and in co-owned PCT WO 2009/035791 (Mar. 19, 2009), the disclosures of which are incorporated by reference herein for the purposes of describing these antibodies, their methods of preparation and their methods of use. Antibodies were diluted to a 3.75 mg/ml working solution in sterile PBS pH 7.4, 0.01% Tween 20 (PBST) and stored at 4° C.

Thirty-six male C57B1/6 mice, at 8-10 weeks of age, were used in this experiment. They were maintained at 20±2° C., at a relative humidity of 55±5%, with a 14 hour light/10 hour dark cycle. On day 0, thirty mice were anaesthetized with an intraperitoneal injection of Nembutal™. A 6 mg/ml solution was used and the injection volume (in microliters) corresponded to ten time the body weight of the animal in grams. Under anaesthesia, the pupils were dilated by topical administration of one drop of Tropicol™ (from a 5 mg/ml stock solution). Photocoagulation was accomplished using an argon laser (532 nm) to place three burns (at 9-, 12- and 3-o′ clock) on the retina using a slit lamp delivery system. The laser was set for a duration of 0.01 sec at an energy of 400 mW, to generate a 50 um burn spot. Production of the spot was confirmed by the observation of a bubble, signifying rupture of the Bruch's membrane.

Animals that had undergone photocoagulation were divided into three groups of ten. One group received 0.75 mg of anti-LOX antibody immediately after photocoagulation and every two days thereafter. A second group received 0.75 mg of anti-LOXL2 antibody immediately after photocoagulation and every two days thereafter. The third group received 200 ul of PBST (vehicle) immediately after photocoagulation and every two days thereafter. Antibody solutions or vehicle were administered intraperitoneally in a volume of 0.2 ml.

Six naïve animals (i.e., animals that did not undergo photocoagulation) were also included in the study.

At day 35 after photocoagulation, all animals were sacrificed by cervical dislocation and both eyes were removed and enucleated. (One animal in the anti-LOX treatment group died on day 16 of the study.) One eye from each of the 35 animals was fixed in 4% paraformaldehyde and embedded in paraffin. Seven micrometer sections were cut and were analyzed to determine degree of inflammation, extent of neovascularization and degree of fibrosis, as follows.

The degree of inflammation was quantitated by determining the area of the section exhibiting CD45 immunoreactivity and expressing this as a percentage of the total area of the lesion, as described in Example 3.

Thin sections were subjected to immunohistochemistry for CD31, a blood vessel marker, whose presence is indicative of neovascularisation. For this analysis, trypsin digestion was conducted for 7 minutes at 37° C., and rabbit serum was used as a blocking agent. Sections were incubated overnight at room temperature with rat anti-mouse CD31 antibody ( 1/500; Pharmingen). The following day the slides were incubated with a biotinylated rabbit anti-rat antibody (Dakocytomation) at a 1/300 dilution for 45 minutes at room temperature. Sections were then developed using a TSA Cyan 3 System (Perkin Elmer TSATM; NEL704A) at room temperature, and washed with TNT washing buffer. Streptavidin peroxidase was used at a 1/100 dilution and cyan 3 was diluted 1/50 in working buffer. The extent of neovascularizaton was quantitated by determining the area of the section exhibiting CD31 immunoreactivity and expressing this as a percentage of the total area of the lesion.

The extent of fibrosis was scored quantitatively by determining the area of the section occupied by collagen fibers (determined by Sirius Red staining) and expressing this area as a percentage of the total area of the lesion.

Methods for measurement and quantitation are described infra in Example 1.

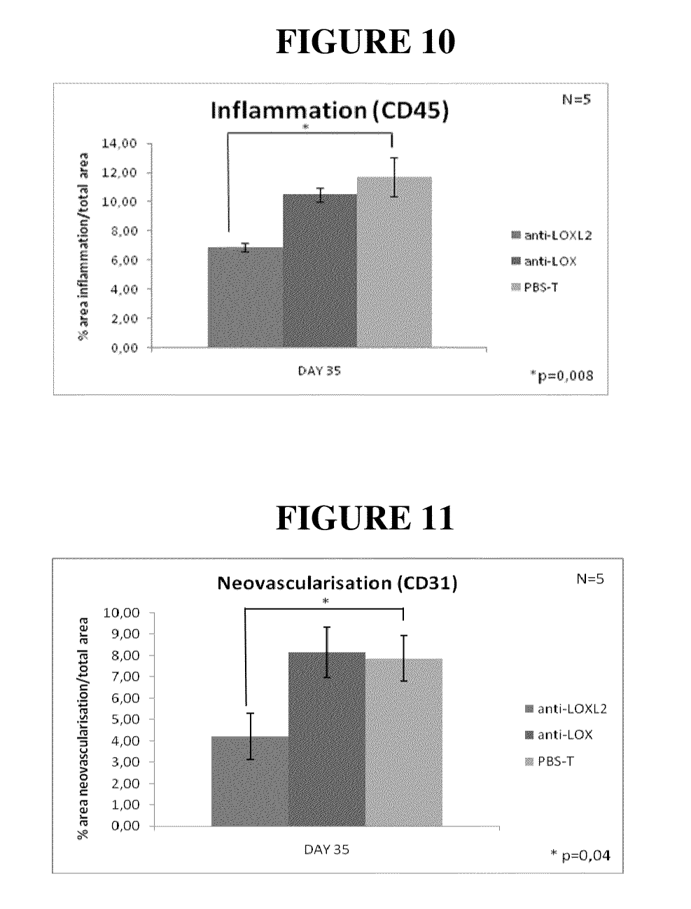

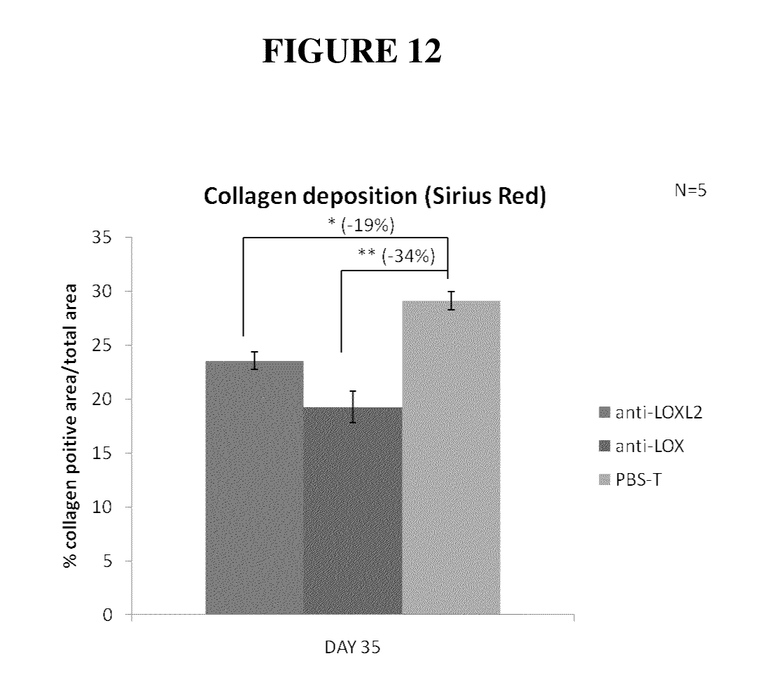

The results of these analyses are shown in FIGS. 10-12. FIG. 10 shows that inflammation, as measured by the CD45-positive area of the lesions, was reduced in subjects treated with an anti-LOXL2 antibody. Similarly, the degree of neovascularization, measured by the CD31-positive area or the lesions, was reduced in subjects that had been treated with an anti-LOXL2 antibody (FIG. 11). Fibrosis, as measured by collagen density, was reduced by both anti-LOX and anti-LOXL2 antibodies (FIG. 12).

What is claimed is:

1. A method for the treatment of ocular neovascularization in an organism, wherein the method comprises inhibiting the activity of a lysyl oxidase-type enzyme in one or more cells of the organism.

2. The method of claim 1, wherein inhibiting comprises binding of an antibody to a lysyl oxidase-type protein.

3. The method of claim 1, wherein the lysyl oxidase-type protein is lysyl oxidase (LOX).

4. The method of claim 1, wherein the lysyl oxidase-type protein is lysyl oxidase-related protein 2 (LOXL2).

5. The method of claim 1, wherein the method further comprises inhibiting the activity of an angiogenic factor in one or more cells of the organism.

6. The method of claim 5, wherein the activity of the angiogenic factor is inhibited by binding of an antibody to the angiogenic factor.

7. The method of claim 5, wherein the angiogenic factor is a vascular endothelial growth factor (VEGF).

8. The method of claim 7, wherein the VEGF is vascular endothelial growth factor A (VEGF-A).

9. The method of claim 1, wherein the ocular neovascularization occurs in a disease selected from the group consisting of age-related macular degeneration (AMD), diabetic retinopathy (DR) and retinopathy of prematurity.

10. The method of claim 2, wherein the antibody is introduced into the eye of the organism.

11. The method of claim 6, wherein the antibodies are introduced into the eye of the organism.

12. The method of claim 2, wherein a polynucleotide encoding the antibody is introduced into the eye of the organism.

13. The method of claim 6, wherein one or more polynucleotides encoding the antibodies are introduced into the eye of the organism.

14. The method of claim 10, wherein the antibody is introduced into one or more retinal epithelial cells.

15. The method of claim 11, wherein the antibodies are introduced into one or more retinal epithelial cells.

16. The method of claim 12, wherein the polynucleotide is introduced into one or more retinal epithelial cells.

17. The method of claim 13, wherein the polynucleotide or polynucleotides are introduced into one or more retinal epithelial cells.

18. The method of claim 12, wherein the polynucleotide is encapsidated in a viral vector selected from the group consisting of adeno-associated virus (AAV), adenovirus and lentivirus.

19. The method of claim 13, wherein the polynucleotide or polynucleotides are encapsidated in a viral vector selected from the group consisting of adeno-associated virus (AAV), adenovirus and lentivirus.

20. The method of claim 18, wherein the viral vector is an adeno-associated virus (AAV).

21. The method of claim 19, wherein the viral vector is an adeno-associated virus (AAV).

22. The method of claim 20, wherein the viral vector is AAV Type 2 or AAV Type 4.

23. The method of claim 21, wherein the viral vector is AAV Type 2 or AAV Type 4.

24. The method of claim 1, wherein the organism is a mammal.

25. The method of claim 24, wherein the mammal is a human.
